(19) United States
(12) Patent Application Publication
(10) Pub. No.: US 2019/0247439 A1
(43) Pub. Date: Aug. 15, 2019

(54) REGENERATIVE CELL THERAPY FOR MUSCULOSKELETAL DISORDERS

(71) Applicant: Cytori Therapetics, Inc., San Diego, CA (US)

(72) Inventors: Steven Kesten, San Diego, CA (US); Marc Hedrick, San Diego, CA (US); John Fraser, San Diego, CA (US)

(21) Appl. No.: 16/331,427

(22) PCT Filed: Oct. 7, 2016
(86) PCT No.: PCT/US16/56043

§ 371 Date: Mar. 7, 2019

(51) Int. Cl. A61K 35/28; A61P 19/02

(52) U.S. Cl. CPC A61K 35/28; A61P 19/02

(57) ABSTRACT

Provided herein are methods and compositions for the treatment of musculoskeletal disorders, including disorders of a joint. In particular, provided herein are methods and compositions for the reduction of the incidence of worsening of cartilage loss, for the prevention of the development of full thickness cartilage loss, prevention or slowing of the formation of osteophytes, prevention or slowing of the formation of bone marrow lesions, prevention

### BACKGROUND

Osteoarthritis is a progressively debilitating disease of the joints, and is the common form of arthritis. Osteoarthritis is characterized by joint pain, swelling, and stiffness, and is associated with a lower quality of life and higher healthcare utilization. While the etiology of arthritis is not fully understood, age, genetics, and trauma to the knee have been shown to be important determinants of risk. Even though osteoarthritis is highly prevalent, disabling and costly, development of therapies capable of arresting structural progression has been slow, with no disease modifying agents approved to date.

Cartilage loss in individuals with osteoarthritis occurs more rapidly than in non-osteoarthritic individuals. It has been shown that the annual rate of loss of total tibial cartilage is between 4.4% and 6.2% in individuals with symptomatic knee osteoarthritis, which is nearly twice the rate of loss in healthy subjects. Worsening in cartilage thickness and cartilage surface area are independently associated with osteoarthritis progression. See, Collins, et al. (2016) Arthrit. Rheum. April 25 doi: 10.1002/art.39731. Accordingly, the prevention or slowing of the progression of cartilage loss and/or minimization of loss of cartilage surface area are potential targets for disease modifying therapies.

Osteophytes, or bone spurs, are bony projections that form along joint margins. Osteophytes are often formed in osteoarthritic joints as a result of damage and wear. Osteophytes usually limit joint movement and typically cause pain. Surgical removal of osteophytes is applied as part of the treatment of osteoarthritis. See, Shin et al (2012), Knee Surg & Relat Res 24(4):187-192. To date, no therapies exist on the market for modifying osteophyte formation. Rather, available treatments include anti-inflammatory medications, and muscle relaxant pain medications, rest and rehabilitation therapy, steroid injections, and surgery. There is an unmet need for therapies to reduce the formation and progression of osteophyte formation.

Bone marrow lesions have been shown to increase the risk of cartilage loss, increase osteoarthritis progression assessed by X-ray, and increase the development of knee pain. Studies have shown that bone marrow lesions were significantly associated with pain on climbing stairs in subjects with symptomatic early knee osteoarthritis. Ip et al. (2011) J Rheumatol. 38:1079-1085. Bone marrow lesions were present in 11% of subjects without osteoarthritis, in 38% with pre-radiographic osteoarthritis and in 71% with radiographic osteoarthritis. Changes in bone marrow lesions are associated with fluctuations in knee pain in the same direction in patients with knee osteoarthritis longitudinally. See, Zhang, et al., (2011) Arthritis Rheum 63:691-699. Studies have shown that pain resolution occurred more frequently when bone marrow lesions became smaller, suggesting that for new treatments and prevention strategies for the symptomatic management of osteoarthritis. To date, there are no therapeutics that target prevention of the formation and/or growth of bone marrow lesions in osteoarthritis. There is an unmet need for therapies to reduce the formation and progression of osteophyte formation.

It has long been known that regenerative cells, e.g., from bone marrow and/or adipose tissue, are capable of differentiating into chondrocytes. It has also been shown that regenerative cells can secrete anti-inflammatory cytokines and factors. For this reason, groups have investigated the use of culture-expanded adipose-derived stem cells, in conjunction with platelet rich plasma (a substance also known to possess anti-inflammatory properties) as potential therapy for regenerating cartilage and reducing inflammation in osteoarthritic joints. Koh, et al. (2013) Arthroscopy 29: 748-55.

### SUMMARY

Described herein are methods and compositions based, in part, on the surprising discovery that treatment of damaged joints, e.g., osteoarthritic joints, with regenerative cells resulted in an improvement in prevention of cartilage loss, preventing or slowing the surface area of the knee exhibiting cartilage loss. The embodiments disclosed herein are also based in part on the surprising discovery that treatment of damaged joints (e.g., osteoarthritic joints) with regenerative cells, prevented or slowed the formation of osteophytes in the joints, and prevented and/or slowed bone marrow lesions in the joints.

Accordingly, in some embodiments, provided is a method of reducing the incidence of worsening of cartilage loss in at least one region of a joint in a subject in need thereof, including the step of identifying a subject with cartilage damage in at least one joint, and administering to the subject a therapeutically effective amount of regenerative cells to the subject. Also provided are regenerative cells for reducing the incidence of worsening of cartilage loss in at least one region of a joint, wherein the joint has cartilage damage. In some embodiments, the at least one region of the joint with is osteoarthritic. In some embodiments, the regenerative cells are injected in the joint space, or formulated for injection into the joint space. In some embodiments, the regenerative cells prevent full thickness cartilage loss. In some embodiments, the regenerative cells prevent partial thickness cartilage loss. In some embodiments, regenerative cells prevent full and/or partial thickness cartilage loss.

In some embodiments, provided is a method of preventing the development of full thickness cartilage loss at a region in a joint that is at risk of developing full thickness cartilage loss, including identifying a subject at risk of developing full thickness cartilage loss in at least one region of a joint and administering to the subject a therapeutically effective amount of regenerative cells. Also provided are regenerative cells for the prevention of the development of full thickness cartilage loss at a region in a joint that is at risk of developing full thickness cartilage loss. In some embodiments, the region in the joint that is at risk of developing full thickness cartilage loss is osteoarthritic. In some embodiments, the region in the joint that is at risk of developing full thickness cartilage loss has no cartilage loss prior to administration of the regenerative cells. In some embodiments, the region in the joint that is at risk of developing full thickness cartilage loss has less than or equal to 10% cartilage loss (but not zero), prior to administration of the regenerative cells. In some embodiments, the region in the joint that is at risk of developing full thickness cartilage loss has between 10% and 75% cartilage loss, prior to administration of the regenerative cells. In some embodiments, the region in the joint that is at risk of developing full thickness cartilage loss has more than 75% cartilage loss, prior to administration of the regenerative cells. In some embodiments, the regenerative cells prevent the development of full thickness cartilage loss for at least 24 weeks, at least 48 weeks, at least 52 weeks, at least 104 weeks, or longer, or within a range defined by any two of the aforementioned time periods following administration of the regenerative cells.

In some embodiments, provided are methods for preventing or slowing the formation of bone marrow lesions in a joint in a subject in need thereof, including identifying a subject having or at risk of developing one or more bone marrow lesions and administering a therapeutically effective amount of regenerative cells to the subject. Also provided are regenerative cells for preventing or slowing the formation of bone marrow lesions in a joint. In some embodiments, the joint is osteoarthritic. In some embodiments, the regenerative cells are injected in the joint space, or formulated for injection into the joint space. In some embodiments, the regenerative cells prevent an increase in size of an existing bone marrow lesion. In some embodiments, the regenerative cells prevent or minimize an increase in bone marrow lesion score in the joint. In some embodiments, regenerative cells prevent full and/or partial thickness cartilage loss.

In each of the embodiments above, the regenerative cells can be adipose-derived, bone marrow-derived, placental-derived, Wharton's jelly-derived, amnion-derived, umbilical cord-derived, skin-derived, corneal stroma-derived, muscle-derived, dental pulp derived, or any combination thereof. Preferably, the regenerative cells are adipose-derived, and include adipose-derived stem and progenitor cells. In each of the embodiments above, the regenerative cells can be cryopreserved. The regenerative cells can be uncultured, while in other embodiments, the regenerative cells are cultured. In each of the embodiments above, the regenerative cells can be plastic adherent. In each of the embodiments above, the regenerative cells can be autologous to the subject. In each of the embodiments above, the regenerative cells can be allogeneic to the subject. In each of the embodiments above, the regenerative cells can be administered to, or formulated for administration into or in the proximity of, the joint. For example, in each of the embodiments above, the regenerative cells can be administered by intra-articular injection, or formulated for intra-articular administration.

### BRIEF DESCRIPTION OF THE DRAWINGS

FIG. 1 is a bar graph showing the percentage of regions of the knee that exhibited worsening of full thickness cartilage loss from a baseline of no full thickness cartilage loss in subjects that received adipose-derived regenerative cells and in subjects that received placebo, as described in Example 1, below.

FIG. 2 is a bar graph showing the percentage of regions of the knee that exhibited a worsening of % area with full or partial thickness cartilage loss from a baseline of no full or partial thickness cartilage loss in subjects that received adipose-derived regenerative cells and in subjects that received placebo, as described in Example 1, below.

FIG. 3 is a bar graph showing the percentage of subjects having ≥1, ≥2, ≥3, or ≥4 subregions of the knee exhibit worsening of full thickness cartilage loss in subjects that received adipose-derived regenerative cells and in subjects that received placebo, as described in Example 1, below.

FIG. 4 is a bar graph showing the percentage of subjects with ≥3 and ≥4 subregions of the knee with worsening to full thickness cartilage loss in subjects treated with regenerative cells as described herein and in subjects that received placebo, as described in Example 1, below.

FIG. 5 is a bar graph showing the percentage of subjects with ≥3 subregions exhibiting worsening in the % surface area of full or partial thickness cartilage loss in subjects treated with regenerative cells as described herein and in subjects that received placebo, as described in Example 1, below.

FIG. 6 is a bar graph showing the percentage of subjects showing an increase in the number of subregions within the central/medial subregions of the knee with full thickness cartilage loss in subjects that received adipose-derived regenerative cells and in subjects that received placebo, as described in Example 1, below.

FIG. 7 is a bar graph showing the percentage of subjects showing an increase in the percentage surface area with full or partial thickness cartilage loss in the central/medial subregions of the knee, in subjects that received adipose-derived regenerative cells and in subjects that received placebo, as described in Example 1, below.

FIG. 8 is a bar graph showing the percentage of subjects with an increasing number of subregions within the central/medial subregions of the knee with either full-thickness cartilage loss or increase in the surface area of full or partial thickness cartilage loss in the central/medial subregions of the knee in subjects that received adipose-derived regenerative cells and in subjects that received placebo, as described in Example 1, below.

FIG. 9 is a bar graph showing the percentage of subjects with full thickness cartilage loss in one or more, two or more, three or more, and four or more subregions in the knee that had no full thickness cartilage loss evident at baseline in subjects that received adipose-derived regenerative cells and in subjects that received placebo, as described in Example 1.

FIG. 10 is a bar graph showing the percentage of subjects with a worsening of osteophyte size score in subjects that received adipose-derived regenerative cells and in subjects that received placebo, as described in Example 2.

FIG. 11 is a bar graph showing the percentage of subregions of the knee with a worsening in osteophyte size from a baseline of zero (no osteophytes in the region) in subjects that received adipose-derived regenerative cells and in subjects that received placebo, as described in Example 2.

FIG. 12 is a bar graph showing the percentage of subjects that showed development of osteophytes in regions of the knee that had no osteophytes at baseline, in subjects that received adipose-derived regenerative cells and in subjects that received placebo, as described in Example 2.

FIG. 13 is a bar graph showing the percentage of subjects that had two or more, or three or more subregions of the knee that developed bone marrow lesions that had no bone marrow lesions at baseline, in subjects that received adipose-derived regenerative cells and in subjects that received placebo, as described in Example 3.

FIG. 14 is a bar graph showing the percentage of subjects with a BML score of <2 at baseline that worsened to ≥2 in subjects that received adipose-derived regenerative cells and in subjects that received placebo as described in Example 3.

### DETAILED DESCRIPTION

The embodiments disclosed herein are based, in part, on the surprising discovery of the beneficial effects regenerative cells have in damaged joints. In particular, it was discovered that regenerative cells can function to prevent cartilage loss, or prevent an increase in the surface area affected by cartilage loss in damaged joints. Furthermore, it was discovered that regenerative cells can function to prevent the formation of new osteophytes and slow the growth of osteophytes in damaged joints. It was also discovered that regenerative cells can prevent the formation of new bone marrow lesions, inhibit and/or slow the growth of bone marrow lesions, and minimize the percentage of bone marrow lesions that are non-cyst.

### Definitions

As used herein, the term “about,” when referring to a stated numeric value, indicates a value within plus or minus 10% of the stated numeric value.

As used herein, the term “derived” means isolated from or otherwise purified or separated from. For example, adipose-derived stem and other regenerative cells are isolated from adipose tissue. Similarly, the term “derived” does not encompass cells that are extensively cultured (e.g., placed in culture conditions in which the majority of dividing cells undergo 3, 4, 5 or less, cell doublings), from cells isolated directly from a tissue, e.g., adipose tissue, or cells cultured or expanded from primary isolates. Accordingly, “adipose derived cells,” including adipose-derived stem and other regenerative cells and combinations thereof, refers to cells obtained from adipose tissue, wherein the cells are not extensively cultured, e.g., are in their “native” form as separated from the adipose tissue matrix.

As used herein, a cell is “positive” for a particular marker when that marker is detectable using an art-accepted assay. For example, an adipose derived regenerative cell is positive for, e.g., CD73 because CD73 is detectable on an adipose derived stem or regenerative cell in an amount detectably greater than background (in comparison to, e.g., an isotype control or an experimental negative control for any given assay). A cell is also positive for a marker when that marker can be used to distinguish the cell from at least one other cell type, or can be used to select or isolate the cell when present or expressed by the cell.

As used herein, “regenerative cells” refers to any heterogeneous or homogeneous population of cells obtained using the systems and methods of embodiments disclosed herein, which cause or contribute to complete or partial regeneration, restoration, or substitution of structure or function of an organ, tissue, or physiologic unit or system to thereby provide a therapeutic, structural or cosmetic benefit. Examples of regenerative cells include: adult stem cells, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and/or precursor cells (and their progeny), and/or lymphocytes.

Accordingly, adipose-derived regenerative cells (“ADRCs”) as used herein refers to any heterogeneous or homogeneous cell population that contains one or more types of adipose-derived regenerative cells including adipose-derived stem cells, endothelial cells (including blood and lymphatic endothelial cells), endothelial precursor cells, endothelial progenitor cells, macrophages, fibroblasts, pericytes, smooth muscle cells, preadipocytes, kertainocytes, unipotent and/or multipotent progenitor and precursor cells (and their progeny), and/or lymphocytes. Adipose-derived stem cells, as determined using a cell culture-based assay (CFU-F assay), comprise at least 0.1% of the cellular component of adipose-derived regenerative cells, e.g., comprise at least 0.2%, 0.3%, 0.4%, 0.5%, 0.6%, 0.7%, 0.8%, 0.9%, 1%, 2,%, 3%, 4%, or 5%, or more or an amount that is within a range defined by any two of the aforementioned percentages.

Similarly, “bone marrow-derived regenerative cells” (“BMRCs”) refers to any heterogeneous or homogeneous cell population that contains one or more types of bone marrow-derived regenerative cells including bone marrow-derived stem cells, endothelial cells (including blood and/or lymphatic endothelial cells), endothelial precursor cells, endothelial progenitor cells, macrophages, fibroblasts, pericytes, smooth muscle cells, preadipocytes, keratinocytes, unipotent and/or multipotent progenitor and/or precursor cells (and their progeny), and/or lymphocytes.

In some contexts, the term “progenitor cell” refers to a cell that is unipotent, bipotent, or multipotent with the ability to differentiate into one or more cell types, which perform one or more specific functions and which have limited or no ability to self-renew. Some of the progenitor cells disclosed herein may be pluripotent.

As used herein the phrase “adherent cells” refers to a homogeneous or heterogeneous population of cells, which are anchorage dependent e.g., require attachment to a surface in order to grow in vitro. In the context of the present disclosure “adherent cells” refers to cells that have been exposed to in vitro culture conditions.

In some contexts, the term “adipose tissue-derived cells” refers to cells extracted from adipose tissue that has been processed to separate the active cellular component (e.g., the cellular component that does not include adipocytes and/or red blood cells) from the mature adipocytes and connective tissue. Separation may be partial or full. That is, the “adipose tissue-derived cells” may or may not contain some adipocytes and connective tissue and may or may not contain some cells that are present in aggregates or partially disaggregated form (for example, a fragment of blood or lymphatic vessel comprising two or more cells that are connected by extracellular matrix) and may or may not contain some red blood cells. This fraction is referred to herein as “adipose tissue-derived cells,” “adipose derived cells,” “adipose derived regenerative cells” or “ADC.” Typically, ADC refers to the pellet of cells obtained by washing and separating the cells from the adipose tissue. The pellet is typically obtained by concentrating a suspension of cells released from the connective tissue and adipose tissue matrix. By way of example, the pellet can be obtained by centrifuging a suspension of adipose-derived cells so that the cells aggregate at the bottom of a centrifuge container, e.g., the stromal vascular fraction. In some embodiments, the adipose-derived cell populations described herein include, among other cell types, leukocytes. In some embodiments, the adipose-derived cell populations described herein include, among other regenerative cell types, endothelial cells.

In some contexts, the term “adipose tissue” refers to a tissue containing multiple cell types including adipocytes and vascular cells. Adipose tissue includes multiple regenerative cell types, including adult stem cells (ASCs), endothelial progenitor and precursor cells, pericytes and the like. Accordingly, adipose tissue refers to fat, including the connective tissue that stores the fat.

In some contexts, the term “unit of adipose tissue” refers to a discrete or measurable amount of adipose tissue. A unit of adipose tissue may be measured by determining the weight and/or volume of the unit. In reference to the disclosure herein, a unit of adipose tissue may refer to the entire amount of adipose tissue removed from a subject, or an amount that is less than the entire amount of adipose tissue removed from a subject. Thus, a unit of adipose tissue may be combined with another unit of adipose tissue to form a unit of adipose tissue that has a weight or volume that is the sum of the individual units.

In some contexts, the term “portion” refers to an amount of a material that is less than a whole. A minor portion refers to an amount that is less than 50%, and a major portion refers to an amount greater than 50%. Thus, a unit of adipose tissue that is less than the entire amount of adipose tissue removed from a subject is a portion of the removed adipose tissue.

The term “joint” can refer to any joint in a subject. Exemplary joints embodied herein include, for example, the zygapophyseal joint; carpometacarpal joint, finger; carpometacarpal joint, thumb; coracoclavicular joint; elbow joint; intermetacarpal joint; interphalangeal joints; metacarpophalangeal joint; midcarpal joint; radiocarpal (wrist) joint; radioulnar joint, distal; radioulnar joint, intermediate; radioulnar joint, proximal; shoulder joint; sternoclavicular joint; wrist joint; temporomandibular joint, sternocostal joints; xiphisternal joint; lumbosacral joint; sacroiliac joint; ankle joint; hip joint; interphalangeal joints; knee joint; metatarsophalangeal joint; tarsometatarsal joints, and/or facet joints, and the like. The embodiments disclosed herein are particularly useful in joints that are susceptible to damage, e.g., osteoarthritic damage, including but not limited to the knee, spine, neck, back, thumbs, big toes, hands, and hips; the joints of the hindfoot (e.g., talocalcaneal joint, talonavicular joint, or calcenocuboid joint), the joints of the midfoot (e.g, the metatarsocuneform joint) and/or the great toe (e.g, the first metatarophalangeal joint).

As used herein the term “cartilage loss” refers to any injury in the cartilage of a joint and includes, for example, injury that extends through the full thickness of the cartilage, injury that extends to only partial thickness of the cartilage, and to tears within the cartilage, or the like.

As used herein, the term “osteophyte development” refers to development of osteophytes where there was no osteophyte previously (e.g., prior to treatment), or to the growth or worsening of an existing osteophyte. As used herein, the term “joint damage” refers to a joint exhibiting cartilage loss, osteophyte formation, bone marrow lesions, and/or joint space narrowing, or the like. Joint damage can include, for example, osteoarthritis, rheumatoid arthritis, psoriatic arthritis, fibromyalgia, gout, pseudogout, and/or mechanical injury (e.g., torn cartilage and the like).

### Methods of Treating Joints

The regenerative cells described herein have been shown to advantageously treat joints, e.g., to prevent and/or slow disease progression in damaged joints. “Damaged joint” can refer to osteoarthritic joints, injury, such as fracture, cartilage tears, septic arthritis, strains or sprains, gout, rheumatoid arthritis, osteomyelitits, lupus, infections caused by a virus, and/or chondromalacia patellae.

In some embodiments, the regenerative cells described herein are used to treat a knee joint. Accordingly, some embodiments provide a method of improving or minimizing the worsening of a joint. By way of example only, in some embodiments, the joint is a knee joint and the regenerative cells improve or minimize worsening of the MOAKS score (MRI Osteoarthritis of the Knee Score), e.g., as described in Hunter, et al., (2011) Osteoarthritis Cartilage 19(8): 990-1002; improve or minimize worsening of the WORMS score (Whole Organ Magnetic Resonance Imaging Score), e.g., as described in Peterfy, et al. (2004) Osteoarthritis Cartilage 12(3): 177-190; improve or minimize worsening of the BLOCKS score (Boston-Leeds Osteoarthritits Knee Score), e.g., as described in Hunter, et al. (2008) Ann Rheum Dis 67(2): 206-211; and/or improve or minimize worsening of the CaLS score (Cartilage Lesion Score), e.g., as described in Alizai, et al. (2014) RNSA 271(2): 479-487; or the like. In some cases the improvement or minimized worsening of the joint is assessed radiographically (e.g., by X-ray) or by arthroscopic visualization. In some embodiments, the joint is a knee, and the regenerative cells prevent and/or minimize meniscal extrusion, prevent or minimize Hoffa synovitis, prevent or minimize effusion-synovitis, or the like. In other embodiments, the joint is a hip joint, and the regenerative cells improve or minimize worsening of the HOAMS score (Hip osteoarthritis MRI-scoring), e.g., as described in Roemer, et al. (2011) Osteoarthritis Cartilage 19(8): 946-62; improve or minimize worsening of the hip OA score, e.g., as described in Neumann, et al. (2007) Osteoarthritis Cartilage 15(8):909-17; improve or minimize worsening of the SHOMRI score (scoring of hip osteoarthritis with MRI), e.g., as described in Lee, et al. (2015) J. Magn. Reson. Imaging 41(6): 1549-57, or any combination thereof. By way of example only, in some embodiments, the joint is a hand, and the regenerative cells described herein improve or minimize worsening of the OHOA-MRI score (Oslo Hand Osteoarthritis MRI Score), e.g., as described in Haugen, et al. (2011) Annals Rheum Dis 70(6): 1033-8; or to improve or minimize worsening of the score e.g., as described in Ramonda, et al. (2016) Rheumatol. 5:2079, or the like. In other embodiments, the joint is the spine, and the regenerative cells described herein improve or minimize worsening of the scoring described in, e.g., Pfirmann et al. (2001) Spine. 26 (17): 1873-8, Griffith, et al. (2007) Spine 32(24): E708-12; or the like.

### Cartilage Loss

Provided herein are methods of treating joints, e.g., damaged joints. In some embodiments, provided is a method of increasing cartilage in at least one region of a joint in a subject in need thereof. In some embodiments, provided is a method of reducing the incidence of worsening of cartilage loss in at least one region of a joint in a subject in need thereof. In some embodiments, the regenerative cells prevent cartilage loss or reduce the incidence of worsening of cartilage loss in the knee, the hand, the hip, the shoulder, the elbow, the finger, the tope, or the spine, or any combination thereof. Accordingly, in some embodiments, the regenerative cells can reduce the number of regions or subregions in a joint that exhibit worsening of cartilage loss. In some embodiments, a specific subregion that has damage at baseline (e.g., at least some cartilage loss), does not worsen over time, or exhibits a lesser degree of worsening from baseline compared to an untreated subject/untreated joint. In some embodiments, the regenerative cells can reduce or prevent or minimize an increase in percent surface area of the joint (e.g., in a particular region or subregion of the joint) that exhibits some cartilage loss, or minimizes the percentage of surface area of the joint exhibiting cartilage loss (e.g., partial cartilage loss or full cartilage loss, or a tear in the cartilage). In other words, the regenerative cells described herein can advantageously reduce or minimize an increase in the surface area of a joint, or subregion of a joint, with cartilage loss (e.g., partial and/or full thickness cartilage loss, or a cartilage tear).

In some embodiments, the joint is the knee joint, and cartilage loss is assessed in several subregions of the knee. For example, in some embodiments, cartilage loss in the knee is assessed in one or more, or a combination of any two or more subregions of the knee: femur lateral central region, femur lateral posterior region, femur medial posterior region, femur trochlea lateral region, femur trochlea medial region, patella lateral region, patella medial region, tibial lateral anterior region, tibial lateral central region, tibia lateral posterior region, tibia medial anterior region, tibia medial central region, and/or tibia medial posterior region.

Cartilage loss can be scored, for example, using well-established methods in the art, e.g., by imaging (magnetic resonance imaging, CT scan, X-ray, ultrasound, arthroscopic visualization, or the like). By way of example only, cartilage loss can be scored on a scale of 0 to 3, wherein a score of 0 refers to no observable cartilage loss; 1 refers to less than 10% cartilage loss; 2 refers to 10%-75% cartilage loss; and 3 refers to >75% cartilage loss. In some embodiments, the regenerative cells disclosed herein prevent worsening from a score of 0 to a score of 1. In some embodiments, the regenerative cells disclosed herein prevent worsening from a score of 0 to a score of 2. In some embodiments, the regenerative cells disclosed herein prevent worsening from a score of 0 to a score of 3. In some embodiments, the regenerative cells disclosed herein prevent worsening from a score of 1 to a score of 2. In some embodiments, the regenerative cells disclosed herein prevent worsening from a score of 1 to a score of 3. In some embodiments, the regenerative cells disclosed herein reduce the number of subregions in a joint that exhibit worsening of cartilage loss. For example, in some embodiments, the regenerative cells can function such that the number or percentage of subregions in a joint exhibiting worsening of cartilage loss will remain the same (e.g., will not increase/worsen). In this regard, the number of subregions that worsen can be reduced by 5%, 10%, 15%, 20%, 25%, 30%, 35%, 40%, or less, or within a range defined by any two of the aforementioned percentages.

Some embodiments include the step of identifying a subject with cartilage damage in at least one region of a joint. The identification can be made by the subject (e.g., self-identification), by a healthcare professional (e.g., a nurse, a physician, a physician's assistant). Cartilage damage can be assessed by any suitable approach known in the art, including but not limited to X-ray imaging, CT scans, and/or MRI imaging, ultrasound.

### Osteophyte Formation

Musculoskeletal disorders are often associated with the formation of new bone at two main sites: the joint margin (osteophytosis) and ligament and tendon insertions (enthesophyte formation). Osteophytes are strongly associated with osteoarthritis, probably forming in response to abnormal stresses on the joint margin. Osteophytes can also develop as an age related phenomenon, unrelated to any joint disease. Studies have reported an association between the presence of osteophytes and knee pain. See, e.g., Kaukienen et al. (2016) Osteoarthritis Cart. 24:1565-1576; Sowers, et al. (2011) J. Bone Joint Surg. Am. (20122) 93:241-251.

The regenerative cells described herein advantageously prevent, inhibit, or minimize the formation/growth of osteophytes in a joint. In some embodiments, the joint is a knee joint. In some embodiments, the joint is a hand joint. In some embodiments, the joint is a hip joint. In some embodiments, the joint is a finger joint. In some embodiments, the joint is an elbow joint. In some embodiments, the joint is a toe joint. In some embodiments, the joint is part of the spine (e.g., a facet joint). In some embodiments, the methods and compositions provided herein can reduce osteophyte formation or growth at a joint. For example, in some embodiments, the joint is a knee joint, and the regenerative cells can prevent, inhibit, or minimize the formation and/or growth of osteophytes in the knee, e.g., in one or more subregions selected from the following: femur lateral central region, femur lateral posterior region, femur medial central region, femur medial posterior region, femur trochlea lateral region, femur trochlea medial region, tibial subspinous region, patella interior region, patellar superior region, patella medial region, patella lateral region, tibia lateral region, and/or tibia medial region.

For example, in some embodiments, the regenerative cells disclosed herein prevent osteophyte formation. Accordingly, in some embodiments, the regenerative cells reduce the percentage of subregions in a joint that develop osteophytes. In some embodiments, the regenerative cells described herein reduce osteophyte size. In some embodiments, the regenerative cells described herein slow or minimize osteophyte growth. In some embodiments, osteophyte size reflects protuberance (how far the osteophyte extends from the joint) rather than total volume of osteophyte. In some embodiments, osteophyte size reflects the total volume of the osteophyte. In some embodiments, osteophyte size can be scored on a scale of 0 to 3, with 0 corresponding to no osteophyte formation, 1 corresponding to small-sized osteophyte formation, 2 corresponding to medium-sized osteophyte formation, and 3 corresponding to large-sized osteophyte formation.

In some embodiments, a specific subregion of a joint that has one or more osteophytes at baseline does not worsen over time, or exhibits a lesser degree of worsening from baseline (e.g., compared to an untreated joint). Osteophyte formation and size can be scored, for example, using well-established methods in the art, e.g., by imaging (magnetic resonance imaging, CT scan, X-ray, and/or ultrasound). In some embodiments, the regenerative cells disclosed herein prevent worsening of osteophytes from a score of 0 to a score of 1. In some embodiments, the regenerative cells disclosed herein prevent worsening of osteophytes from a score of 0 to a score of 2. In some embodiments, the regenerative cells disclosed herein prevent worsening of osteophytes from a score of 0 to a score of 3, e.g., using a scoring method described in Hunter, et al. (2011) Osteoarthritis Cart. 19(8): 990-1002, or any other art-accepted method. In some embodiments, the regenerative cells disclosed herein prevent worsening of osteophytes from a score of 1 to a score of 2. In some embodiments, the regenerative cells disclosed herein prevent worsening of osteophytes from a score of 1 to a score of 3. In some embodiments, the regenerative cells disclosed herein reduce the number of subregions in a joint that exhibit worsening of osteophytes. For example, in some embodiments, the regenerative cells can function such that the number or percentage of subregions in a joint exhibiting worsening of osteophytes will remain the same (e.g, will not increase/worsen). In this regard, the number of subregions that worsen can be reduced by 5%, 10%, 15%, 20%, 25%, 30%, 35%, 40%, or less, or within a range defined by any two of the aforementioned percentages.

### Bone Marrow Lesions

Bone marrow lesions, or “BMLs”, show up on MRI images as regions of bone beneath the cartilage with ill-defined high signal and are believed to largely represent areas of bone marrow edema, fibrosis, and necrosis. BMLs are described as ill-defined areas of high signal intensity (SI) on T2-weighted, fat saturated or short tau inversion recovery (STIR) Magnetic Resonance images. BMLs are similarly visible by contrast enhanced MRI. Histologically they represent a number of non-characteristic abnormalities including fibrovascular tissue. Bone marrow lesions are known to play a role in pain and in structural progression in subjects with knee osteoarthritis or at risk for osteoarthritis. See, e.g., Guermazi, et al. (2011) Curr Opin. Rheumatol. 23(5): 484-491. The incidence of bone marrow lesions has been associated with an accelerated rate of osteoarthritis progression. For example, in one study it was reported that the space within a joint is lost at a significantly faster rate in subjects with bone marrow lesions than in individuals without bone marrow lesions. See, Edwards et al (2016) J Rheumatol 43(3) 657-65.

The regenerative cells described herein desirably prevent the formation of, and/or reduce the worsening of, bone marrow lesions in a joint. In some embodiments, the joint is a knee. In some embodiments, the joint is the hand. In some embodiments, the joint is a hip. In some embodiments, the joint is a shoulder. In some embodiments, the joint is an elbow. In some embodiments, the joint is a finger. In some embodiments, the joint is a toe. In some embodiments, the joint is the spine.

In some embodiments, the joint is a knee joint, and bone marrow lesions are assessed in the femur lateral central region, femur lateral posterior region, femur medial central region, femur medial posterior region, femur trochlea lateral region, femur trochlea medial region, tibial subspinous region, patella interior region, patellar superior region, patella medial region, patella lateral region, tibia lateral region, and/or tibia medial region, or any combination thereof. In some embodiments, the regenerative cells described herein prevent the formation of bone marrow lesions, e.g., in joints or subregions of joints in which no bone marrow lesions are detected. In some embodiments, the regenerative cells described herein prevent or minimize an increase in the number of bone marrow lesions present in a joint, or a subregion of a joint, e.g. a joint with no, or one or more BMLs.

In some embodiments, the regenerative cells described herein reduce or minimize the increase in size of a BML in a sub-region of a joint, as a percentage of the sub-regional volume. In some embodiments, the size of BML can be scored on a scale of 0 to 3. For example, 0 can refer to no BML. 1 can refer to BML that is <33% of the subregional volume. 2 can refer to BML that is 33-66% of the subregional volume. 3 can refer to BML that is >66% of the subregional volume. Accordingly, in some embodiments, the regenerative cells described herein can prevent BML scores from progressing from 0 to 1, 0 to 2, or 0 to 3. In some embodiments, the regenerative cells described herein can prevent BML scores from progressing from 1 to 2, or 1 to 3. In some embodiments, the regenerative cells described herein can prevent BML scores from progressing from 2 to 3. In some embodiments, the regenerative cells decrease the percentage of subregions in a joint that show an increase in size of BML. In this regard, the number of subregions that worsen can be reduced by 5%, 10%, 15%, 20%, 25%, 30%, 35%, 40%, or less, or within a range defined by any two of the aforementioned percentages.

In some embodiments, the regenerative cells described herein can minimize or prevent the percentage of the BML size that is BML from increasing. For example, in some embodiments, the % of BML size that is BML (versus cyst), can be scored on a scale of 0 to 3. For example, a score of 0 can refer to no % of BML size that is BML (versus cyst); 1 can refer to 33% of BML size that is BML (versus cyst); 2 can refer to 33-66% of BML size that is BML (versus cyst) and 3 can refer to >66% of BML size that is BML (versus cyst). Accordingly, in some embodiments, the regenerative cells described herein can prevent BML scores from progressing from 0 to 1, 0 to 2, or 0 to 3. In some embodiments, the regenerative cells described herein can prevent BML scores from progressing from 1 to 2, or 1 to 3. In some embodiments, the regenerative cells described herein can prevent BML scores from progressing from 2 to 3. In some embodiments, the regenerative cells decrease the percentage of subregions in a joint that show worsening of % BML size that is BML. In this regard, the number of subregions that worsen can be reduced by 5%, 10%, 15%, 20%, 25%, 30%, 35%, 40%, or less, or within a range defined by any two of the aforementioned percentages. In some embodiments, the regenerative cells decrease the percentage of subregions in a joint that show worsening of number of BMLs in that region. Some embodiments relate to methods of preventing or minimizing an increase in bone marrow lesion score in a joint of a subject in need thereof. In some embodiments, bone marrow lesion scores can be calculated using any art-accepted methods. Exemplary, non-limiting examples of bone marrow scoring can be found in Nielsen, et al. (2014) BMC Musculoskel Disorders 15:447; Peterfy, et al. (2004) Osteoarthr Cartil. 12: 177-190; Kornaat et al. (2005) Skelet Radiol. 34: 95-102; Hunter, et al. (2011) Osteoarthr Cara 19: 990-1002; Hunter et al. (2008) Ann Rheum Dis. 67: 206-211, and the like.

### Methods of Administration

In some embodiments, the methods disclosed herein include administering a therapeutically effective amount of a composition comprising regenerative cells to a subject. As used herein, the term “therapeutically effective amount” refers to an amount sufficient to mitigate or prevent cartilage loss or the progression of cartilage loss in a damaged joint. “Therapeutically effective amount” can also refer to an amount sufficient to mitigate or prevent osteophyte formation or to halt or slow the growth of existing osteophytes in a damaged joint. In some embodiments, “therapeutically effective amount” refers to an amount sufficient to prevent the formation of bone marrow lesions, and/or to prevent the growth of bone marrow lesions, or to minimize the size of non-cyst material in a bone marrow lesion, in a damaged joint. Determination of the exact dose of regenerative cells for the embodiments disclosed herein is well within the ambit of the ordinary skill in the art.

The amount and frequency of administration of the compositions can vary depending on, for example, what is being administered, the state of the patient, and the manner of administration. In therapeutic applications, compositions can be administered to a patient with a damaged joint (e.g., an osteoarthritic joint), in an amount sufficient to relieve or least partially cartilage loss, osteophyte formation, or bone marrow lesions. The dosage is likely to depend on such variables as the type of joint, and extent of the damage to the joint, as well as the age, weight and general condition of the particular subject, and the route of administration. Effective doses can be extrapolated from dose-response curves derived from in vitro or animal model test system.

In some embodiments, at least 1×10² regenerative cells is a therapeutically effective amount. In some embodiments, at least 1×10³ regenerative cells is a therapeutically effective amount. In some embodiments, at least 1×10⁴ cells is a therapeutically effective amount. In some embodiments, at least 1×10⁵ regenerative cells is a therapeutically effective amount. In some embodiments, at least 1×10⁶ regenerative cells is a therapeutically effective amount. In some embodiments, at least 1×10⁷ regenerative cells is a therapeutically effective amount. In some embodiments, at least 1×10⁸ regenerative cells is a therapeutically effective amount. In some embodiments, at least 1×10⁹ regenerative cells is a therapeutically effective amount. In some embodiments, at least 1×10¹⁰ regenerative cells is a therapeutically effective amount. In some embodiments, the amount of regenerative cells provided is within a range defined by any two of the aforementioned amounts of cells. In some embodiments, a greater number of regenerative cells is therapeutically effective to joints with a larger surface area than to treat joints with a smaller surface area. In some embodiments, a greater number of regenerative cells is therapeutically effective to treat joints with more damage than to treat joints with little damage.

In some embodiments, the regenerative cells comprise at least 0.05% stem cells. For example, in some embodiments, the regenerative cells comprise at least 0.1%, 0.2%, 0.3%, 0.4%, 0.5%, 0.6%, 0.7%, 0.8%, 0.9%, 1.0%, 2%, 3%, 4%, 5%, 10%, 15%, 20%, 25%, 50%, or more, stem cells or an amount of cells within a range defined by any two of the aforementioned percentages. That is, in some embodiments, at least 0.1%, 0.2%, 0.3%, 0.4%, 0.5%, 0.6%, 0.7%, 0.8%, 0.9%, 1.0%, 2%, 3%, 4%, 5%, 10%, 15%, 20%, 25%, 50%, or more, of the nucleated cells within the regenerative cell population are stem cells or the amount of nucleated cells within the regenerative cell population that are stem cells are within a range defined by any two of the aforementioned percentages.

### Regenerative Cells

In the embodiments disclosed herein, regenerative cells are used for treating damaged joints, e.g., to prevent or inhibit cartilage loss, to prevent and/or slow the formation of osteophytes, and to prevent and/or slow the formation of bone marrow lesions.

As mentioned above, a population of “regenerative cells” disclosed herein can be a homogeneous or heterogeneous population of cells that cells that which cause or contribute to complete or partial regeneration, restoration, or substitution of structure or function of an organ, tissue, or physiologic unit or system to thereby provide a therapeutic, structural or cosmetic benefit. Examples of regenerative cells include, but are not limited to adult stem cells, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and/or multipotent progenitor and precursor cells (and their progeny), and/or lymphocytes.

The regenerative cells disclosed herein can be isolated from various tissues, including, but not limited to bone marrow, placenta, adipose tissue, skin, eschar tissue, endometrial tissue, adult muscle, corneal stroma, dental pulp, Wharton's jelly, amniotic fluid, and/or umbilical cord. The regenerative cells disclosed herein can be isolated from the tissues above using any means known to those skilled in the art or discovered in the future.

By way of example only, regenerative cells can be isolated from adipose tissue by a process wherein tissue is excised or aspirated. Excised or aspirated tissue can be washed, and then enzymatically or mechanically disaggregated in order to release cells bound in the adipose tissue matrix. Once released, the cells can be suspended. By way of example only, regenerative cells useful in the embodiments disclosed herein can be isolated using the methods and/or devices described in U.S. Pat. Nos. 7,390,484; 7,585,670, 7,687,059, 8,309,342, 8,440,440, US Patent Application Publication Nos. 2013/0164731, 2013/0012921, 2012/0164113, US2008/0014181. International Patent Application Publication No. WO2009/073724, WO/2013030761, and the like, each of which is herein incorporated by reference.

Exemplary, non-limiting methods for isolation of regenerative cells from bone marrow useful in the embodiments disclosed herein are described in U.S. Pat. No. 5,879,940, U.S. Patent Application Publication Nos 2013/0101561, 2013/0266541 European Patent Application Publication No. EP2488632A1, EP0241578A2, EP2624845A2, International Patent Application Publication No. WO2011047289A1, WO1996038482A, each of which is herein incorporated by reference.

Exemplary, non-limiting methods for isolation of regenerative cells from placental tissue useful in the embodiments disclosed herein are described in U.S. Pat. No. 8,580,563, U.S. Patent Application Publication No. 20130040281, International Patent Application Publication No. WO2003089619A, Klein, et al. (2011) Methods Mol Biol. 698:75-88, Vellasamy, et al. (2012) World J Stem Cells 4(6): 53-61; Timmins, et al. (2012) Biotechnol Bioeng. 109(7):1817-26; Semenov, et al. (2010) Am J Obstet Gynecol 202:193-e.13, and the like, each of which is herein incorporated by reference.

Exemplary, non-limiting methods for isolation of regenerative cells from skin useful in the embodiments disclosed herein are described in Toma, et al. (2001), Nat Cell Biol. 3(9):778-84; Nowak, et al. (2009), Methods Mol Biol. 482:215-32; U.S Patent Application Publication No. 2007/0248574, and the like, each of which is herein incorporated by reference.

Exemplary, non-limiting methods for isolation of regenerative cells from eschar tissue useful in the embodiments disclosed herein are described in Van der Veen, et al. (2012), Cell Transplant. 21(5):933-42, and elsewhere herein below.

Exemplary, non-limiting methods for isolation of regenerative cells from endometrial tissue useful in the embodiments disclosed herein are described in U.S. Patent Application Publication No. 2013/0156726, 2008/0241113, and the like, each of which is herein incorporated by reference in its entirety.

Exemplary, non-limiting methods for isolation of regenerative cells from muscle tissue useful in the embodiments disclosed herein are described in U.S. Pat. No. 6,337,384, U.S. Patent Application Publication No. 2001/019966, 2011/0033428, 2005/0220775, and the like, each of which is herein incorporated by reference.

Exemplary, non-limiting methods for isolation of regenerative cells from corneal tissue useful in the embodiments disclosed herein are described in U.S. Patent Application Publication No. 2005084119, Sharifi, et al. (2010) Biocell. 34(1):53-5, and the like, each of which is herein incorporated by reference.

Exemplary, non-limiting methods for isolation of regenerative cells from dental pulp useful in the embodiments disclosed herein are described in U.S. Patent Application Publication No. 2012/0251504, Gronthos, et al. (2011) Methods Mol Biol. 698:107-21; Suchánek, et al. Acta Medica (Hradec Kralove). 2007; 50(3):195-201; Yildirm, Sibel, “Isolation Methods of Dental Pulp Stem Cells,” in Dental Pulp Stem Cells: Springer Briefs in Stem Cells, pp. 41-51, © 2013, Springer New York, N.Y., N.Y., and the like, each of which is herein incorporated by reference.

Exemplary, non-limiting methods for isolation of regenerative cells from Wharton's jelly useful in the embodiments disclosed herein are described in U.S. Patent Application Publication Nos. 2013/0183273, 2011/0151556, International Patent Application Publication No. WO 04/072273A1, Sheshareddy, et al. (2008) Methods Cell Biol. 86:101-19, Mennan, et al. (2013) BioMed Research International, Article ID 916136, Corotchi, et al. (2013) Stem Cell Research & Therapy 4:81, and the like, each of which is herein incorporated by reference.

Exemplary, non-limiting methods for isolation of regenerative cells from amniotic fluid useful in the embodiments described herein are described in U.S. Pat. No. 8,021,876, International Patent Application Publication No. WO 2010/033969A1, WO 2012/014247A1, WO 2009/052132, U.S. Patent Application Publication No. 2013/0230924, 2005/0054093, and the like, each of which is herein incorporated by reference.

Exemplary, non-limiting methods for isolation of regenerative cells from the umbilical cord useful in the embodiments described herein are described in U.S. Patent Application Publication No. 20130065302, Reddy, et al. (2007), Methods Mol Biol. 407:149-63, Hussain, et al. (2012) Cell Biol Int. 36(7):595-600, Pham, et al. (2014) Journal of Translational Medicine 2014, 12:56, Lee, et al. (2004) Blood 103(5): 1669-1675, and the like, each of which is herein incorporated by reference.

The regenerative cells in the methods and compositions described herein can be a heterogeneous population of cells that includes stem and other regenerative cells. In some embodiments, the regenerative cells in the methods and compositions described herein can include stem and/or endothelial precursor cells. In some embodiments, the regenerative cells can include stem and/or pericyte cells. In some embodiments, the regenerative cells can include stem cells and/or leukocytes. For example, in some embodiments, the regenerative cells can include stem cells and/or macrophages. In some embodiments, the regenerative cells can include stem cells and/or M2 macrophages. In some embodiments, the regenerative cells can include pericytes and/or endothelial precursor cells. In some embodiments, the regenerative cells can include platelets. Preferably, the regenerative cells comprise stem cells and endothelial precursor cells. In some embodiments, the regenerative cells can include regulatory cells such as Regulatory T cells (Treg cells).

In some embodiments, the regenerative cells are adipose-derived. Accordingly, some embodiments provide methods and compositions for mitigating or reducing cartilage loss, mitigating or reducing osteophyte formation, or mitigating or reducing bone marrow lesions with adipose-derived regenerative cells, e.g., that include adipose-derived stem and endothelial precursor cells.

In some embodiments, the regenerative cells are not cultured prior to use. By way of example, in some embodiments, the regenerative cells are for use following isolation from the tissue of origin, e.g., bone marrow, placenta, adipose tissue, skin, eschar tissue, endometrial tissue, adult muscle, cornea stroma, dental pulp, Wharton's jelly, amniotic fluid, umbilical cord, and the like.

In some embodiments, the regenerative cells are cultured prior to use. For example, in some embodiments, the regenerative cells are subjected to “limited culture,” i.e., to separate cells that adhere to plastic from cells that do not adhere to plastic. Accordingly, in some embodiments, the regenerative cells are “adherent” regenerative cells. An exemplary, non-limiting method of isolating adherent regenerative cells from adipose tissue are described e.g., in Zuk, et al. (2001). Exemplary, non-limiting method of isolating adherent regenerative cells from bone marrow are described, e.g., Pereira (1995) Proc. Nat. Acad. Sci. USA 92:4857-4861, Castro-Malaspina et al. (1980), Blood 56:289-30125; Piersma et al. (1985) Exp. Hematol. 13:237-243; Simmons et al., 1991, Blood 78:55-62; Beresford et al., 1992, J. Cell. Sci. 102:341-351; Liesveld et al. (1989) Blood 73:1794-1800; Liesveld et al., Exp. Hematol 19:63-70; Bennett et al. (1991) J. Cell. Sci. 99:131-139), U.S. Pat. No. 7,056,738, and the like.

In some embodiments, the regenerative cells are cultured for more than 3 passages in vitro. For example, in some embodiments, the regenerative cells are cultured for 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more passages in vitro or within a range defined by any two of the aforementioned number of passages.

The regenerative cells described herein can be cultured according to approaches known in the art, and the cultured cells can be used in several of the embodied methods. For example, regenerative cells can be cultured on collagen-coated dishes or 3D collagen gel cultures in endothelial cell basal medium in the presence of low or high fetal bovine serum or similar product, as described in Ng, et al., (2004), Microvasc. Res. 68(3):258-64, incorporated herein by reference. Alternatively, regenerative cells can be cultured on other extracellular matrix protein-coated dishes. Examples of extracellular matrix proteins that may be used include, but are not limited to, fibronectin, laminin, vitronectin, and collagen IV. Gelatin or any other compound or support, which similarly promotes adhesion of endothelial cells into culture vessels may be used to culture regenerative cells, as well.

Examples of basal culture medium that can be used to culture regenerative cells in vitro include, but are not limited to, EGM, RPMI, M199, MCDB131, DMEM, EMEM, McCoy's 5A, Iscove's medium, and/or modified Iscove's medium, or any other medium or blend of media known in the art to support the growth of blood endothelial cells. In some embodiments, the regenerative cells are cultured in EGM-2MV media. Examples of supplemental factors or compounds that can be added to the basal culture medium that could be used to culture regenerative cells include, but are not limited to, ascorbic acid, heparin, endothelial cell growth factor, endothelial growth supplement, glutamine, HEPES, Nu serum, fetal bovine serum, human serum, equine serum, plasma-derived horse serum, iron-supplemented calf serum, penicillin, streptomycin, amphotericin B, basic and acidic fibroblast growth factors, insulin-growth factor, astrocyte conditioned medium, fibroblast or fibroblast-like cell conditioned medium, sodium hydrogencarbonate, epidermal growth factor, bovine pituitary extract, magnesium sulphate, isobutylmethylxanthine, hydrocortisone, dexamethasone, dibutyril cyclic AMP, insulin, transferrin, sodium selenite, oestradiol, progesterone, growth hormone, angiogenin, angiopoietin-1, Del-1, follistatin, granulocyte colony-stimulating factor (G-CSF), erythropoietin, hepatocyte growth factor (HGF)/scatter factor (SF), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular endothelial growth factor (VEGF)/vascular permeability factor (VPF), interleukin-3 (IL-3), interleukin 7 (IL-7), interleukin-8 (IL-8), ephrins, and/or matrix metalloproteinases (such as MMP2 and MMP9), or any other compound known in the art to promote survival, proliferation or differentiation of endothelial cells.

Further processing of the cells may also include: cell expansion (of one or more regenerative cell types) and/or cell maintenance (including cell sheet rinsing and media changing); sub-culturing; cell seeding; transient transfection (including seeding of transfected cells from bulk supply); harvesting (including enzymatic, non-enzymatic harvesting and harvesting by mechanical scraping); measuring cell viability; cell plating (e.g., on microtiter plates, including picking cells from individual wells for expansion, expansion of cells into fresh wells); high throughput screening; cell therapy applications; gene therapy applications; tissue engineering applications; therapeutic protein applications; viral vaccine applications; harvest of regenerative cells or supernatant for banking or screening, measurement of cell growth, lysis, inoculation, infection or induction; generation of cell lines (including hybridoma cells); culture of cells for permeability studies; cells for RNAi and viral resistance studies; cells for knock-out and transgenic animal studies; affinity purification studies; structural biology applications; assay development and/or protein engineering applications.

In some embodiments, methods for isolating regenerative useful in the embodiments described herein can include positive selection (selecting the target cells), negative selection (selective removal of unwanted cells), or combinations thereof. In addition to separation by flow cytometry as described herein and in the literature, cells can be separated based on a number of different parameters, including, but not limited to, charge or size (e.g., by dielectrophoresis or various centrifugation methods, etc.).

By way of example, the regenerative cells useful in the methods of treatment disclosed herein may be identified by different combinations of cellular and genetic markers. For example, in some embodiments, the regenerative cells express CD90. In some embodiments, the regenerative cells do not express significant levels of lin. In some embodiments, the regenerative cells do not express significant levels of ckit. In some embodiments, the regenerative cells are CD90+/lin−/ckit−/CD45-.

In some embodiments, the regenerative cells express STRO-1. In some embodiments, the regenerative cells express STRO-1 and/or CD49d. In some embodiments, the regenerative cells express STRO-1, CD49d, and one or more of CD29, CD44, CD71, CD90, C105/SH2 and/or SH3. In some embodiments, the regenerative cells express STRO-1, CD49d, and one or more of CD29, CD44, CD71, CD90, C105/SH2 and/or SH3, but express low or undetectable levels of CD106.

In some embodiments, the regenerative cells express one or more of STRO-1, CD49d, CD13, CD29, SH3, CD44, CD71, CD90, and/or CD105, or any combination thereof. By way of example only, in some embodiments, the regenerative cells express each of do not express significant levels of CD31, CD34, CD45 and/or CD104 and do not express detectable levels of CD4, CD8, CD11, CD14, CD16, CD19, CD33, CD56, CD62E, CD106 and/or CD58.

In some approaches, the regenerative cells are CD14 positive and/or CD11b positive. In some embodiments, the cells are depleted for cells expressing the markers CD45(+). In some embodiments, the cells are depleted for cells expressing glycophorin-A (GlyA). In some embodiments, the cells are depleted for CD45(+) and GlyA(+) cells.

Negative selection of cells, e.g., depletion of certain cell types from a heterogeneous population of cells can done using art-accepted techniques, e.g., utilizing micromagnetic beads or the like. In some embodiments, the regenerative cells are CD34+.

In some embodiments, the regenerative cells are not cryopreserved. In some embodiments, the regenerative cells are cryopreserved. For example, in some embodiments, the regenerative cells include cryopreserved cells, e.g., as described in Liu, et al. (2010) Biotechnol Prog. 26(6):1635-43, Carvalho, et al. (2008) Transplant Proc.; 40(3):839-41, International Patent Application Publication No. WO 97/039104, WO 03/024215, WO 2011/064733, WO 2013/020492, WO 2008/09063, WO 2001/011011, European Patent No. EP0343217 B1.

In some embodiments, regenerative cells are isolated from a subject with a joint with damaged cartilage, e.g., an osteoarthritic joint.

### Combination Therapy

As explained in further detail below, some embodiments provide for treatment of subjects with combination therapy, i.e., one or more additional additives (e.g., pharmaceutical agents, biologic agents, or other therapeutic agents) in addition to the regenerative cells as described herein.

In some embodiments, the one or more additional “agents” described above can be administered in a single composition with the regenerative. In some embodiments, the one or more additional “agents” can be administered separately from the regenerative cells. For example, in some embodiments, one or more additional agents can be administered just prior to, or just after, administration of the regenerative cells. As used herein, the term “just prior” can refer to within 15 minutes, 30 minutes, an hour, 2 hours, 3 hours, 4 hours, 5 hours, or within a range defined by any two of the aforementioned times. Likewise, the phrase “just after administration” can refer to within 15 minutes, 30 minutes, an hour, 2 hours, 3 hours, 4 hours, 5 hours, or within a range defined by any two of the aforementioned times.

Additional agents useful in combination therapy in the methods described herein include, for example, growth factors, cytokines, platelet rich plasma, steroids, non-steroidal anti-inflammatory agents, anti-bacterial and/or anti-fungal agents, as well as other agents known in the art to have beneficial effects in treatment of burn.

1) Growth Factors, Cytokines, and Hormones

Various growth factors, cytokines, and hormones have been shown to have beneficial effects, e.g., in re-epithelialization and recovery in burn injury. See, e.g., Wenczak, et al. (1992) J. Clin. Invest. 90:2392-2401.

In some embodiments, subjects can be administered one or more growth factors, cytokines or hormones, including combinations thereof, in addition to the regenerative cells disclosed herein. For example, in some embodiments, growth factors are administered concomitantly with, prior to, or following the administration of the regenerative cells. Non-limiting examples of growth factors useful in the embodiments disclosed herein include, but are not limited to, angiogenin, angiopoietin-1 (Ang-1), angiopoietin-2 (Ang-2), brain-derived neurotrophic factor (BDNF), Cardiotrophin-1 (CT-1), ciliary neurotrophic factor (CNTF), Del-1, acidic fibroblast growth factor (aFGF), basic fibroblast growth factor (bFGF), follistatin, ganulocyte colony-stimulating factor (G-CSF), glial cell line-derived neurotrophic factor (GDNF), hepatocyte growth factor (HGF), scatter factor (SF), Interleukin-8 (IL-8), leptin, midkine, nerve growth factor (NGF), neurotrophin-3 (NT-3), Neurotrophin-4/5, Neurturin (NTN), placental growth factor, Platelet-derived endothelial cell growth factor (PD-ECGF), Platelet-derived growth factor-BB (PDGF-BB), Pleiotrophin (PTN), Progranulin, Proliferin, PBSF/SDF-1, Transforming growth factor-alpha (TGF-alpha), Transforming growth factor-beta (TGF-beta), Tumor necrosis factor-alpha (TNF-alpha), Vascular endothelial growth factor (VEGF), and/or vascular permeability factor (VPF), erythropoietin (see, e.g., Tobalem, et a. (2012) Br. J. Surg. 99(9):1295-1303).

2) Anti-Inflammatory Agents

In some embodiments, subjects are administered one or more anti-inflammatory agents, in addition to the regenerative cells as disclosed herein. As used herein, the term “anti-inflammatory agent” refers to any compound that reduces inflammation, and includes, but is not limited to steroids, non-steroidal anti-inflammatory drugs, and other biologics that have been demonstrated to have an anti-inflammatory effect.

Accordingly, in some embodiments, steroids are administered concomitantly with, prior to, or following the administration of the regenerative cells. Non-limiting examples of steroids useful in the embodiments disclosed herein include, but are not limited to, progestegens, e.g., progesterone, and the like; corticosteroids, e.g., prednisone, aldosterone, cortisol, androgens, e.g., testosterone, and estrogens.

Other anti-inflammatory agents useful in the embodiments disclosed herein include, for example, antibodies that inhibit action of TNF-α, IL-6 (see, e.g., Sun, et al. (2012) Repair and Regeneration, 20(4): 563-572), or anti-TNF conjugates, Sun, et al. (2012) Wound Repair Regen. 20(4): 563-572. These anti-inflammatory agents have been demonstrated to exhibit beneficial effects in burn recovery.

Non-steroidal anti-inflammatory drugs useful in the embodiments disclosed herein include propionic derivatives; acetic acid derivatives; biphenylcarboxylic acid derivatives; fenamic acid derivatives; and/or oxicams. Examples of anti-inflammatory actives include without limitation acetaminophen, diclofenac, and/or diclofenac sodium and other salts, ibuprofen and its salts, acetaminophen, indomethacin, oxaprozin, pranoprofen, benoxaprofen, bucloxic acid, and/or elocon; and mixtures thereof.

3) Anti-Oxidants

In some embodiments, the methods and compositions disclosed herein include administration of one or more anti-oxidants in addition to the regenerative cells. Antioxidants useful in the embodiments disclosed herein include, but are not limited to, N-acetylcysteine, curcumarin, galactomannan, and/or pyruvate and/or other alpha-ketoacids, thioglycollate, and/or vitamin A and derivatives, including retinoic acid, retinyl aldehyde, retin A, retinyl palmitate, adapalene, and/or beta-carotene; vitamin B (panthenol, provitamin B5, panthenic acid, and/or vitamin B complex factor); vitamin C (ascorbic acid and salts thereof) and derivatives such as ascorbyl palmitate; vitamin D including calcipotriene (a vitamin D3 analog), vitamin E including its individual constituents alpha-, beta-, gamma-, delta-tocopherol, and/or cotrienols and/or mixtures thereof and/or vitamin E derivatives including vitamin E palmitate, vitamin E linolate and/or vitamin E acetate; vitamin K and derivatives; and/or vitamin Q (ubiquinone) and any combination thereof.

4) Platelet-Containing Fluids

In some embodiments, subjects are administered platelet rich plasma, in addition to the regenerative cells disclosed herein. For example, in some embodiments, a platelet containing fluid is administered concomitantly with, prior to, or following the administration of the regenerative cells. In some embodiments, the regenerative cells as disclosed herein are combined with a synergistically effective amount of platelet-containing fluid.

As used herein, the term “platelet-containing fluid” refers to any fluid, either biological or artificial, which contains platelets, Non-limiting examples of such fluids include various forms of whole blood, blood plasma, platelet rich plasma, concentrated platelets in any medium, or the like, derived from human and non-human sources. For example, in some embodiments, the platelet-containing fluid refers to blood, platelets, serum, platelet concentrate, platelet-rich plasma (PRP), platelet-poor plasma (PPP), plasma, and/or fresh frozen plasma. (FFP).

The term “PRP” as used herein refers to a concentration of platelets greater than the peripheral blood concentration suspended in a solution of plasma. Methods for isolating PRP useful in the embodiments disclosed herein are known in the art. See, e.g., U.S. Pat. No. 8,557,535, International Patent Application Publication No. WO 09/155069, U.S. Patent Application Publication Nos, US20100183561, US20030060352, US20030232712, US20130216626, US20130273008, US20130233803, US20100025342, European Patent No. EP1848474B1, and the like. Platelets or PRP can be suspended in an excipient other than plasma. In some embodiments, the platelet composition can include other excipients suitable for administration to a human or non-human animal including, but not limited to isotonic sodium chloride solution, physiological saline, normal saline, dextrose 5% in water, dextrose 30% in water, lactated ringer's solution and the like. Typically, platelet counts in PRP as defined herein range from 500,000 to 1,200,000 per cubic millimeter, or even more. PRP may be obtained using autologous, allogeneic, or pooled sources of platelets and/or plasma. PRP may be obtained from a variety of animal sources, including human sources. In preferred embodiments, PRP according to the invention is buffered to physiological pH.

### Methods of Administration

Compositions administered according to the methods described herein can be introduced into the subject by, e.g., by injection, e.g., by intra-articular, intravenous, intraarterial, intradermal, intramuscular, intra-lymphatic, intranodal, intramammary, intraperitoneal, intrathecal, retrobulbar, intrapulmonary (e.g., term release); by oral, sublingual, nasal, anal, vaginal, or transdermal delivery, and/or by surgical implantation at a particular site. The introduction may consist of a single dose or a plurality of doses over a period of time. In such cases the plurality of introductions need not be by the same mechanism. For example, in some embodiments introduction at one time might be in the form of an intra-articular injection of the regenerative cells whereas at another time the administration may be intravascular. Vehicles for cell therapy agents are known in the art and have been described in the literature. See, for example Remington's Pharmaceutical Sciences, 18th Ed. (1990, Mack Publ. Co, Easton Pa. 18042) pp 1435-1712, incorporated herein by reference. Sterile solutions are prepared by incorporating the regenerative cells that in the required amount in the appropriate buffer with or without various of the other components described herein.

In some embodiments, the regenerative cells described herein can be administered directly to a joint, e.g., a damaged joint, an osteoarthritic joint, or the like. For example, in some embodiments, the regenerative cells disclosed herein are formulated for injection. Accordingly, in some embodiments, the compositions disclosed herein are formulated for intra-articular, intravenous, intraarterial, intradermal, intramuscular, intraperitoneal, intrasternal, subcutaneous, intranodal and intra-lymphatic injection, infusion, and/or placement. In some embodiments, the compositions disclosed herein are formulated for intra-lymphatic delivery. Accordingly, in some embodiments, the regenerative cells can be injected into a joint, e.g., intra-articularly, or the like. In some embodiments, the regenerative cells are injected into the synovial fluid of the joint. In some embodiments, the regenerative cells are injected into the tissues comprising or adjacent to the joint (e.g., into the synovium, into the fat pad, into tendons and/or ligaments, into adjacent skin, subcutaneous space, and/or muscle).

In some embodiments, the regenerative cells disclosed herein injected via subcutaneous or intramuscular injection, adjacent to a joint, e.g., a damaged joint, an osteoarthritic joint, or the like. In some embodiments, the regenerative cells are formulated for administration in multiple doses, e.g., in multiple injections in and/or around a joint, e.g., a damaged joint, an osteoarthritic joint, or the like. In some embodiments, the number of injections depends upon the size of joint. For example, in some embodiments, as the area (and/or the severity) of the joint, e.g., joint with damage, or joint with osteoarthritis, increases, a greater the number of injections of the regenerative cells is provided. In some embodiments, for example, the regenerative cells as disclosed herein are injected into and around a joint every 0.1 mm², 0.2 mm², 0.3 mm², 0.4 mm², 0.5 mm², 0.6 mm², 0.7 mm², 0.8 mm², 0.9 mm², 1.0 mm², 2 mm², 3 mm², 4 mm², 5 mm², 6 mm², 7 mm², 8 mm², 9 mm², 10 mm², 20 mm², 30 mm², 40 mm², 50 mm², 60 mm², 70 mm², 80 mm², 90 mm², 1 cm², 5 cm², 10 cm², 20 cm², 30 cm², 40 cm², 50 cm², 60 cm², 70 cm², 80 cm², 90 cm², or 100 cm² or within an area that is within a range defined by any two of the aforementioned values. The skilled artisan will readily appreciate that various devices, e.g., the JUVAPEN™ injection device (Juvaplus, SA, Switzerland), etc., suitable for the injection of multiple doses of regenerative cells, can be used in the administration of the regenerative cells according to the embodiments disclosed herein. In some embodiments, the regenerative cells are formulated for delivery in a single injection, e.g., a single intra-articular injection.

In some embodiments, the regenerative cells disclosed herein can be administered via one or multiple intravenous injections. For example, in some embodiments, the regenerative cells can be administered via a single intravenous infusion over a period of 1 min, 2 min, 3 min, 4 min, 5 min, 10 min, 30 min, 45 min, 1 h, 2 h, or longer or within a range defined by any two of the aforementioned time points.

In some embodiments, the regenerative cells disclosed herein can be administered by applying the cells to a scaffold as discussed elsewhere herein (e.g., including but not limited to biocompatible synthetic and non-synthetic matrices, such as skin substitutes), and applying the scaffold seeded with the regenerative cells to a joint. In some embodiments, a scaffold (e.g., including but not limited to biocompatible synthetic and non-synthetic matrices, such as skin substitutes) is applied to the burn, and the regenerative cells disclosed herein are applied onto the scaffold.

In some embodiments, the compositions including the regenerative cells disclosed herein are administered within 5 min, 10 min, 15 min, 20 min, 30 min, 40 min, 50 min, 1 h, 2 h, 3 h, 4 h, 5 h, 6 h, 7 h, 8 h, 9 h, 10 h, 11 h, 12 h, 24 h, 36 h, 48 h, 60 h, 1 week, 2 weeks, or less (but not zero) or within a range defined by any two of the aforementioned times, following an injury. In some embodiments, the regenerative cells are administered serially over a period of time (e.g., wherein the subject can be administered regenerative cells in a single or in a plurality of doses each time). For example, in some embodiments, the regenerative cells described herein can be administered every 12 hours, every day, every 2, 3, 4, 5, 6, 7, 8, 9, 10, 11, 12, 13, 14 days, every month, every six months, annually, or more or within a range defined by any two of the aforementioned times. The frequency of treatment may also vary. The subject can be treated one or more times per day (e.g., once, twice, three, four or more times) or every so-many hours (e.g., about every 2, 4, 6, 8, 12, or 24 hours or within a range defined by any two of the aforementioned times). The time course of treatment may be of varying duration, for example, for two, three, four, five, six, seven, eight, nine, ten or more days or within a range defined by any two of the aforementioned times. For example, the treatment can be twice a day for three days, twice a day for seven days, twice a day for ten days or within a range defined by any two of the aforementioned times. While our expectation is that the treatment will continue as the patients tissues go through a healing and/or remodeling process, treatment cycles can be repeated at intervals. For example treatment can be repeated weekly, bimonthly or monthly, and the periods of treatment can be separated by periods in which no treatment is given. The treatment can be a single treatment or can last as long as the life span of the subject (e.g., many years).

As disclosed herein, the regenerative cells can be provided to the subject, or applied directly to damaged tissue, or in proximity to the damaged tissue, without further processing or following additional procedures to further purify, modify, stimulate, or otherwise change the cells after isolation from the tissue of origin. For example, the cells obtained from a patient may be provided back to said patient without culturing the cells before administration. In several embodiments, the collection and processing of adipose tissue, as well as, administration of the regenerative cells is performed at a patient's bedside. In a preferred embodiment the regenerative cells are extracted from the tissue of the person into whom they are to be implanted (i.e., are autologous), thereby reducing potential complications associated with antigenic and/or immunogenic responses to the transplant. In some embodiments, the cells extracted from or derived from another individual, e.g., are allogeneic.

Additional Alternatives

Several preferred alternatives of the invention described herein are provided below.

1. A method of reducing worsening of cartilage loss in at least one region of a joint in a subject in need thereof, comprising:
identifying a subject at risk of developing new or worsened cartilage loss in at least one region of a joint; and
administering to the subject a therapeutically effective amount of regenerative cells.

2. The method of alternative 1, wherein the joint is selected from the group consisting of a knee, a hand, a hip, an elbow, a finger, a spine, an ankle, and a toe.

3. The method of alternative 2, wherein the joint is a knee.

4. The method of alternative 3, wherein the region of the knee joint is selected from the group consisting of: femur lateral central region, femur lateral posterior region, femur medial posterior region, femur trochlea lateral region, femur trochlea medial region, patella lateral region, patella medial region, tibial lateral anterior region, tibial lateral central region, tibia lateral posterior region, tibia medial anterior region, tibia medial central region, and tibia medial posterior region.

5. The method of anyone of alternatives 1-4, wherein the reduction of the worsening of cartilage loss in at least one region of a joint comprises reduction of worsening of full thickness cartilage loss.

6. The method of anyone of alternatives 1-4, wherein the reduction of the worsening of cartilage loss in at least one region of a joint comprises reduction of worsening of partial thickness cartilage loss.

7. The method of anyone of alternatives 1-4, wherein reduction of the worsening of cartilage loss in at least one region of a joint comprises reduction of worsening of full and/or partial thickness cartilage loss.

8. A method of reducing development of full thickness cartilage loss at a region of a joint that is at risk of developing full thickness cartilage loss, comprising:
identifying a subject at risk of developing full thickness cartilage loss in at least one region of a joint; and
administering to the subject a therapeutically effective amount of regenerative cells.

9. The method of alternative 8, wherein the joint is selected from the group consisting of a knee, a hand, a hip, an elbow, a finger, a spine, an ankle, and a toe.

10. The method of alternative 9, wherein the joint is a knee.

11. The method of alternative 10, wherein the region of the knee is selected from the group consisting of: femur lateral central region, femur lateral posterior region, femur medial posterior region, femur trochlea lateral region, femur troclea medial region, patella lateral region, patella medial region, tibial lateral anterior region, tibial lateral central region, tibia lateral posterior region, tibia medial anterior region, tibia medial central region, and tibia medial posterior region.

12. The method of anyone of alternatives 8-11, wherein the subject has at least one region of the joint with no full thickness cartilage loss prior to administration of the regenerative cells.

13. The method of anyone of alternatives 8-11, wherein the region at risk of development of full thickness cartilage loss has no full thickness cartilage loss prior to administration of the regenerative cells.

14. The method of anyone of alternatives 8-11, wherein the region at risk of development of full thickness cartilage loss has less than 10% full thickness cartilage loss prior to administration of the regenerative cells.

15. The method of anyone of alternatives 8-11, wherein the region at risk of development of full thickness cartilage loss has between 10 to 75% full thickness cartilage loss prior to administration of the regenerative cells.

16. The method of anyone of alternatives 8-11, wherein the region at risk of development of full thickness cartilage loss has greater than 75% full thickness cartilage loss prior to administration of the regenerative cells.

17. The method of anyone of alternatives 8-11, wherein the prevention of the development of full thickness cartilage loss comprises prevention of full thickness cartilage loss for at least 24 weeks.

18. The method of anyone of alternatives 8-11, wherein the prevention of the development of full thickness cartilage loss comprises prevention of full thickness cartilage loss for at least 48 weeks.

19. The method of anyone of alternatives 8-11, wherein the prevention of the development of full thickness cartilage loss comprises a reduction in the surface areas of full cartilage loss in a region of the knee.

20. A method of reducing development of partial thickness cartilage loss at a region of a joint that is at risk of developing partial thickness cartilage loss, comprising:
identifying a subject at risk of developing partial thickness cartilage loss in at least one region of a joint; and
administering to the subject a therapeutically effective amount of regenerative cells.

21. The method of alternative 20, wherein the joint is selected from the group consisting of a knee, a hand, a hip, an elbow, a finger, a spine, an ankle, and a toe.

22. The method of alternative 21, wherein the joint is a knee.

23. The method of alternative 22, wherein the region of the knee is selected from the group consisting of: femur lateral central region, femur lateral posterior region, femur medial posterior region, femur trochlea lateral region, femur trochlea medial region, patella lateral region, patella medial region, tibial lateral anterior region, tibial lateral central region, tibia lateral posterior region, tibia medial anterior region, tibia medial central region, and tibia medial posterior region.

24. The method of anyone of alternatives 20-23, wherein the subject has at least one region of the joint with no partial thickness cartilage loss prior to administration of the regenerative cells.

25. The method of anyone of alternatives 20-23, wherein the region at risk of development of partial thickness cartilage loss has no partial thickness cartilage loss prior to administration of the regenerative cells.

26. The method of anyone of alternatives 20-23, wherein the region at risk of development of partial thickness cartilage loss has less than 10% partial thickness cartilage loss prior to administration of the regenerative cells.

27. The method of anyone of alternatives 20-23, wherein the region at risk of development of partial thickness cartilage loss has between 10 to 75% partial thickness cartilage loss prior to administration of the regenerative cells.

28. The method of anyone of alternatives 20-23, wherein the region at risk of development of partial thickness cartilage loss has greater than 75% partial thickness cartilage loss prior to administration of the regenerative cells.

29. The method of anyone of alternatives 20-23, wherein the prevention of the development of partial thickness cartilage loss comprises prevention of partial thickness cartilage loss for at least 24 weeks.

30. The method of anyone of alternatives 20-23, wherein the prevention of the development of partial thickness cartilage loss comprises prevention of partial thickness cartilage loss for at least 48 weeks.

31. The method of anyone of alternatives 20-23, wherein the prevention of the development of partial thickness cartilage loss comprises a reduction in the surface areas of cartilage loss in a region of the knee.

32. A method of preventing or slowing the formation and/or growth of osteophytes in one or more regions of a joint, comprising:
identifying a subject having, or at risk of developing, one or more osteophytes in one or more regions of a joint; and
administering to the subject a therapeutically effective amount of regenerative cells.

33. The method of alternative 32, further comprising assessing osteophytes in the subject.

34. The method of alternative 32, wherein the one or more regions of joint has no osteophyte prior to administration of the therapeutically effective amount of regenerative cells.

35. The method of alternative 32, wherein the joint is selected from the group consisting of: a knee, a hand, a hip, an elbow, a finger, a spine, an ankle, and a toe.

36. The method of alternative 32, wherein the joint is a knee.

37. The method of alternative 32, wherein the region of the knee is selected from the group consisting of: femur lateral central region, femur lateral posterior region, femur medial central region, femur medial posterior region, femur trochlea lateral region, femur trochlea medial region, tibial subspinous region, patella interior region, patellar superior region, patella medial region, patella lateral region, tibia lateral region, and tibia medial region.

38. A method of preventing or slowing the formation and/or growth of bone marrow lesions in a joint of a subject in need thereof, comprising:
identifying a subject having or at risk of developing one or more bone marrow lesions, and
administering to the subject a therapeutically effective amount of regenerative cells.

39. The method of alternative 38, wherein the joint is selected from the group consisting of: a knee, a hand, a hip, an elbow, a finger, a spine, an ankle, and a toe.

40. The method of alternative 39, wherein the joint is a knee.

41. A method of preventing or slowing an increase in size of an existing bone marrow lesion, comprising:
identifying a subject having or at risk of having a bone marrow lesion in one or more regions of a joint; and
administering to the subject a therapeutically effective amount of regenerative cells.

42. The method of alternative 41, wherein the joint is selected from the group consisting of: a knee, a hand, a hip, an elbow, a finger, a spine, an ankle, and a toe.

43. The method of alternative 42, wherein the joint is a knee.

44. The method of alternative 43, wherein the region of the knee is selected from the group consisting of: femur lateral central region, femur lateral posterior region, femur medial central region, femur medial posterior region, femur trochlea lateral region, femur trochlea medial region, tibial subspinous region, patella interior region, patellar superior region, patella medial region, patella lateral region, tibia lateral region, and tibia medial region.

45. A method of preventing the worsening in percentage of a bone marrow lesion that is not a cyst in a joint of a subject in need thereof, comprising:
identifying a subject having a bone marrow lesion in one or more regions of a joint; and
administering to the subject a therapeutically effective amount of regenerative cells.

46. The method of alternative 45, wherein the joint is selected from the group consisting of: a knee, a hand, a hip, an elbow, a finger, a spine, an ankle, and a toe.

47. The method of alternative 46, wherein the joint is a knee.

48. The method of alternative 47, wherein the region of the knee is selected from the group consisting of: femur lateral central region, femur lateral posterior region, femur medial central region, femur medial posterior region, femur trochlea lateral region, femur trochlea medial region, tibial subspinous region, patella interior region, patellar superior region, patella medial region, patella lateral region, tibia lateral region, and tibia medial region.

49. A method of preventing or minimizing an increase in bone marrow lesion score in a joint of a subject in need thereof, comprising:
identifying a subject having or at risk of having a bone marrow lesion in one or more regions of a joint; and
administering to the subject a therapeutically effective amount of regenerative cells.

50. The method of alternative 49, wherein the joint is selected from the group consisting of: a knee, a hand, a hip, an elbow, a finger, a spine, an ankle, and a toe.

51. The method of alternative 50, wherein the joint is a knee.

52. The method of alternative 51, wherein the region of the knee is selected from the group consisting of: femur lateral central region, femur lateral posterior region, femur medial central region, femur medial posterior region, femur trochlea lateral region, femur trochlea medial region, tibial subspinous region, patella interior region, patellar superior region, patella medial region, patella lateral region, tibia lateral region, and tibia medial region.

53. The method of any one of alternatives 1-51, wherein the regenerative cells are adipose-derived regenerative cells.

54. The method of any one alternatives 1-51, wherein the regenerative cells are not cultured prior to administration to the subject.

55. The method of any one of alternatives 1-51, wherein the regenerative cells are cryopreserved prior to administration to the subject.

56. The method of any one of alternatives 1-51, wherein the administration comprises injection into or in the proximity of the joint space.

57. The method of alternative 56, wherein the joint is a knee, and wherein the administration comprises intra-articular injection.

58. The method of any one of alternatives 1-51, wherein the regenerative cells are autologous.

59. The method of any one of alternatives 1-51, wherein the regenerative cells are allogeneic.

### EXAMPLES

The following examples are provided to demonstrate particular situations and settings in which this technology may be applied and are not intended to restrict the scope of the invention and the claims included in this disclosure.

### Example 1—Minimization of Cartilage Loss in Individuals with Knee Osteoarthritis

A total of 94 patients with osteoarthritis of the knee were enrolled in a double-blind, placebo-controlled, dose escalation study to study the effects of regenerative cell therapy on various aspects of osteoarthritis. Patients with diagnosis of osteoarthritis in one or both knees by the American College of Rheumatology criteria, with imaging findings of degenerative changes in the joint were eligible for the study. All patients underwent small volume liposuction under local anesthesia. The collected lipoaspirate was processed in the Cytori CELUTION® cell processing system to isolate and concentrate adipose-derived regenerative cells for immediate intraartiular administration.

After cell processing, patients were randomized 1:1:1 ratio into the following groups: placebo; low dose (20 million cells); and high dose (40 million cells).

At baseline and again at 48 weeks post-treatment, patients underwent an MRI. Cartilage loss in the following regions of the knee was analyzed: femur lateral central region, femur lateral posterior region, femur medial posterior region, femur trochlea lateral region, femur troclea medial region, patella lateral region, patella medial region, tibial lateral anterior region, tibial lateral central region, tibia lateral posterior region, tibia medial anterior region, tibia medial central region, tibia medial posterior region. A radiologist blinded to treatment examined each region of the knee joint for articular cartilage damage, including the amount of the region affected by full thickness cartilage injury and the amount of the region affected by full or partial thickness cartilage injury. The radiologist scored thickness of cartilage loss (%) on a scale of 0 to 3 as follows:

0=none;

1 less than 10%,

2=10-75%,

3=greater than 75%.

Full or partial cartilage loss as a percentage of surface area was scored on a scale of 0 to 3 as follows:

0=none;

1 less than 10% of regional cartilage surface area

2=10-75% of regional cartilage surface area

3=greater than 75% of regional cartilage surface area.

As shown in Table 1 below, the incidence of regions that exhibited worsening was greater in the placebo group than in the treated group. Specifically, the percentage of subregions of the knee over all patients that received adipose-derived regenerative cell treatment that exhibited worsening was lower than the percentage of subregions of the knee over all patients that received placebo. The data in Table 1 further show that the percentage of subregions of the knee over all patients that received adipose-derived regenerative cell treatment that exhibited worsening of full thickness cartilage loss was lower (3.5%) than the percentage of subregions of the knee over all patients that received placebo (6.9%).

| | | % full Thickness |
|---|---|---|
| | All regions | cartilage loss |
| Placebo | 4.15 (174/4,210) | 6.9% (26/378) |
| Treatment (high and | 3.29% (265/8,044) | 3.5% (26/742) |
| low dose combined) | | |

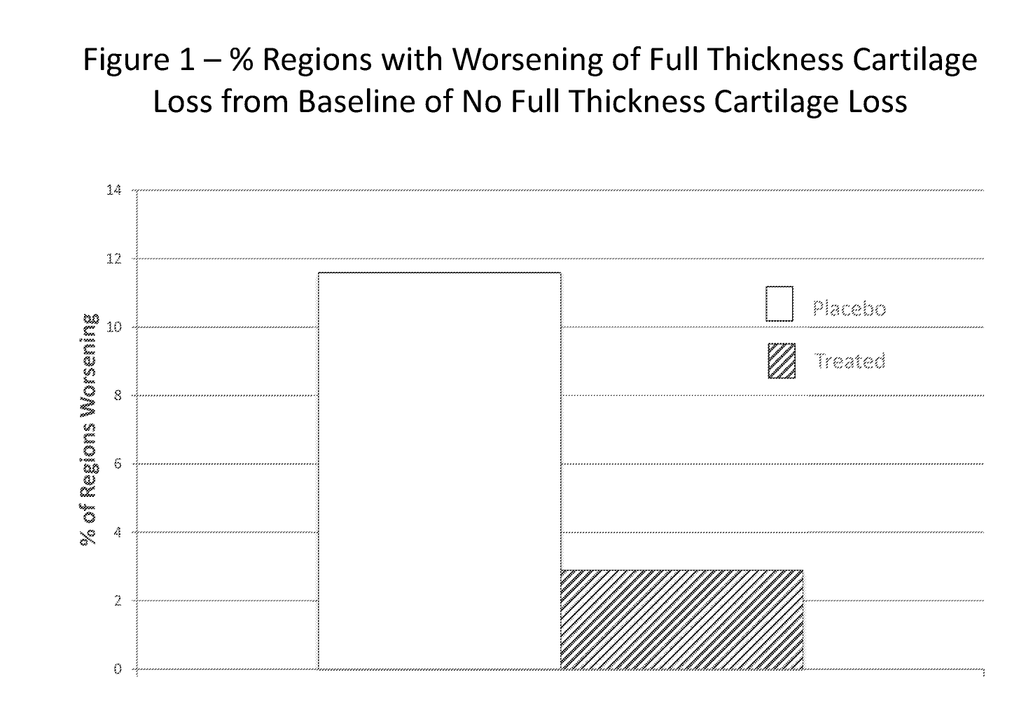

FIG. 1 Shows that the percentage of subregions of the knee that worsened with respect to full thickness cartilage loss from a baseline of no full thickness cartilage loss was less in patients that received adipose-derived regenerative cell treatment that in patients that received placebo.

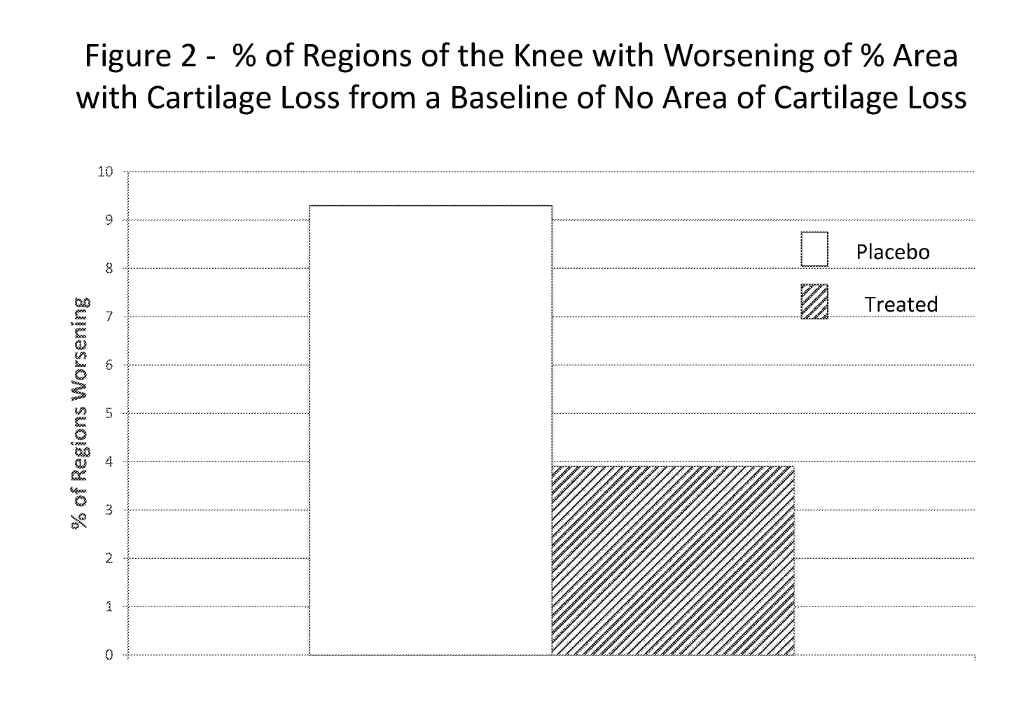

FIG. 2 shows that the percentage of regions in the knee with worsening of the percentage of the region surface area with cartilage loss from a baseline of no cartilage loss was less in subjects that received adipose-derived regenerative cell treatment, as compared to the group receiving placebo.

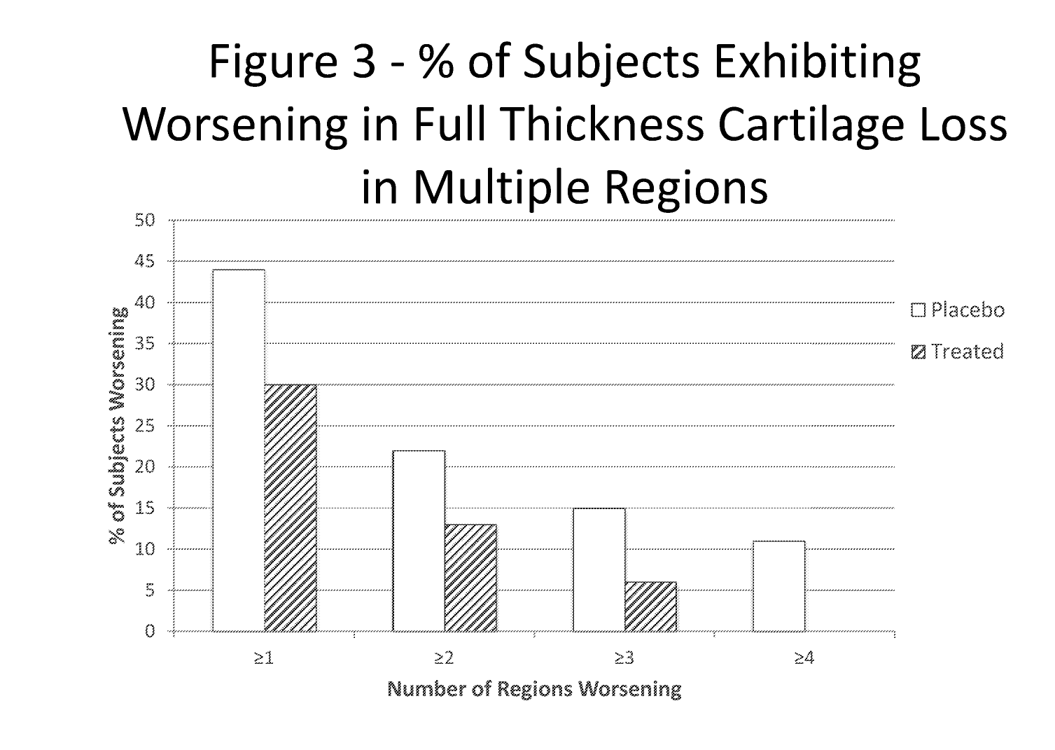

FIG. 3 shows that the treatment group showed reduced incidence of multiple subregions of the knee exhibiting worsening of full thickness cartilage loss (irrespective of baseline level), when compared to the placebo group.

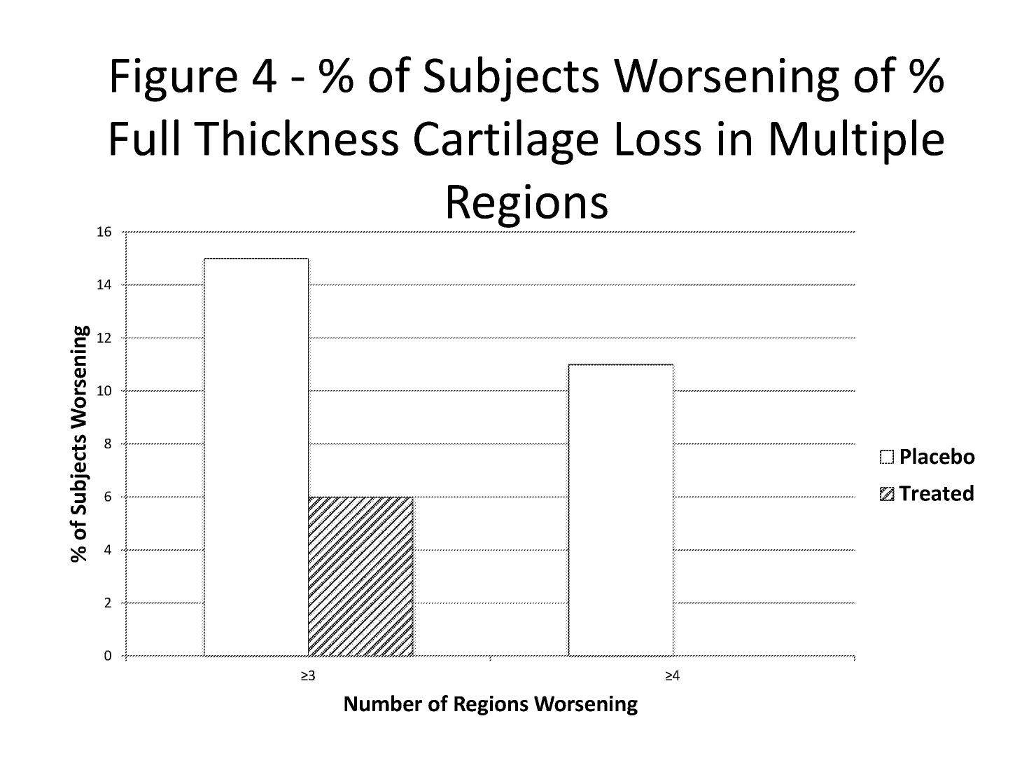

FIG. 4 shows that treatment reduced the percentage of subjects exhibiting a worsening of full thickness cartilage loss in multiple regions.

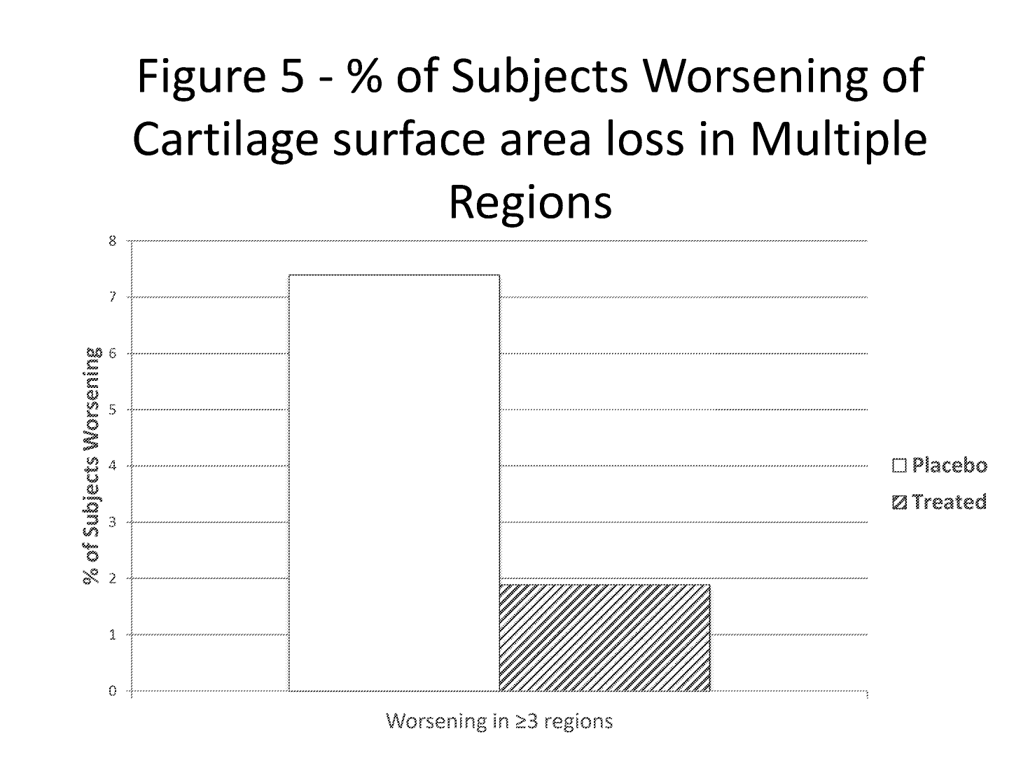

FIG. 5 shows that treatment reduced the percentage of subjects with worsening of the percentage of surface area in three or more subregions, as compared to subjects that received placebo.

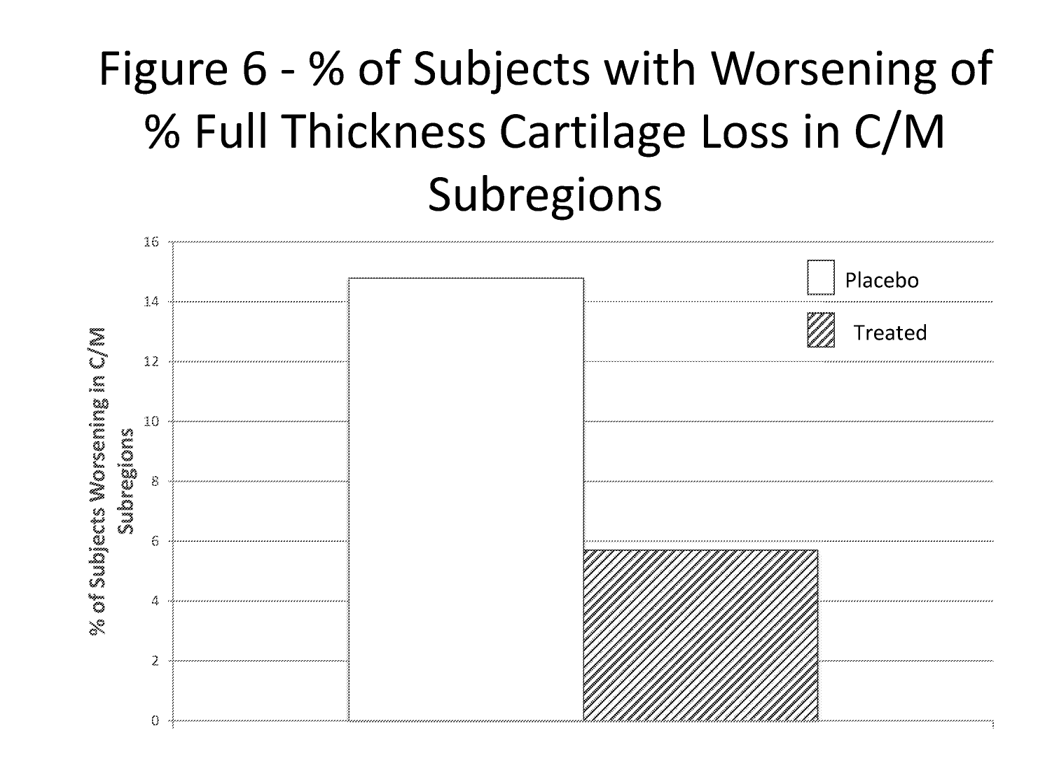

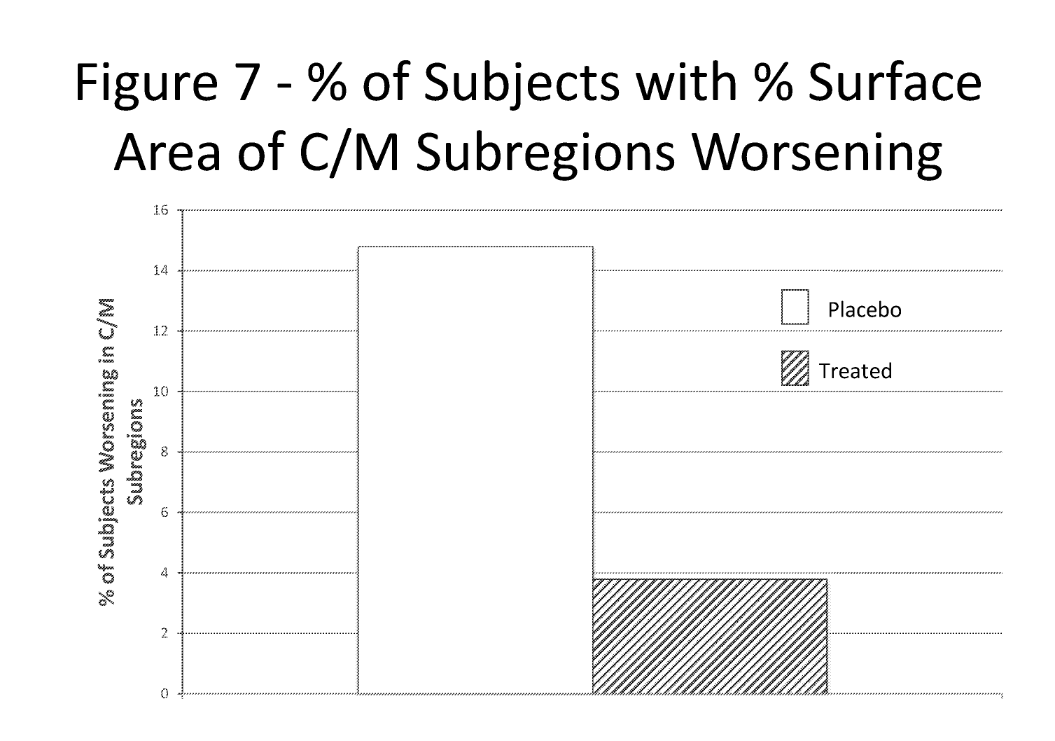

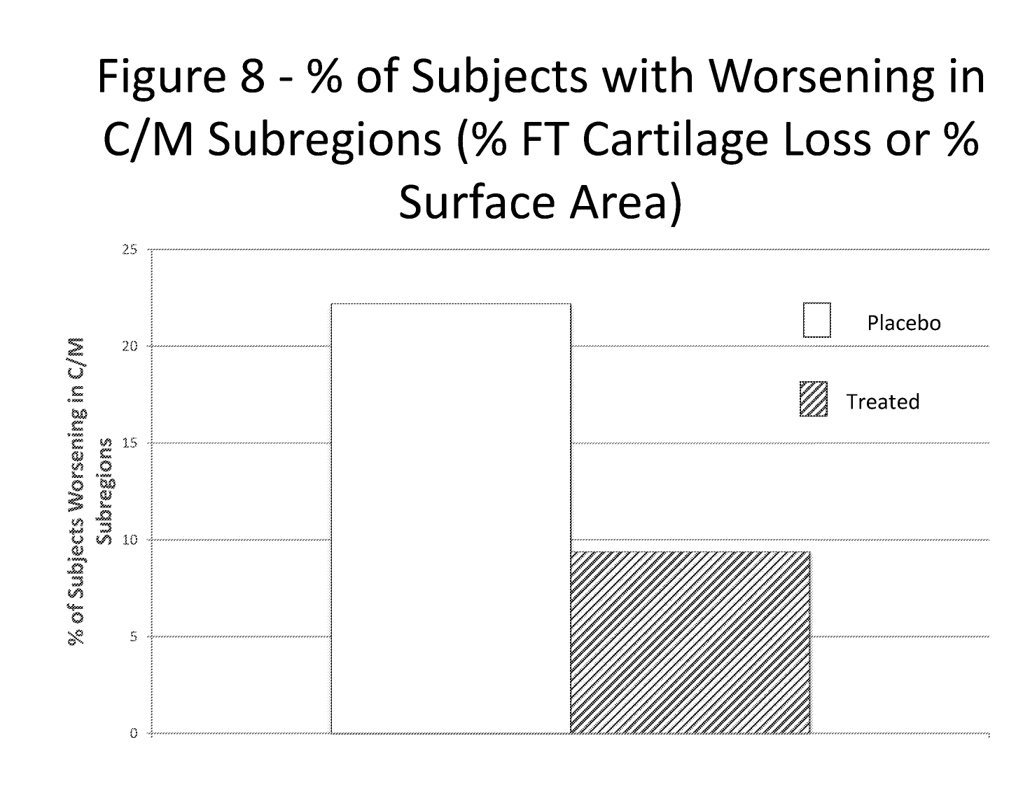

Patients with central/medial cartilage loss a have been reported to be at 1.9× odds ratio greater risk of radiographic progression of osteoarthritis. (Eckstein, et al. (2015) Arthrit. Rheum. 67(12): 3184-3189. FIGS. 6-8 show that the percentage of subjects worsening in central medial subregions was lower in patients treated with adipose-derived regenerative cells compared to patients treated with placebo.

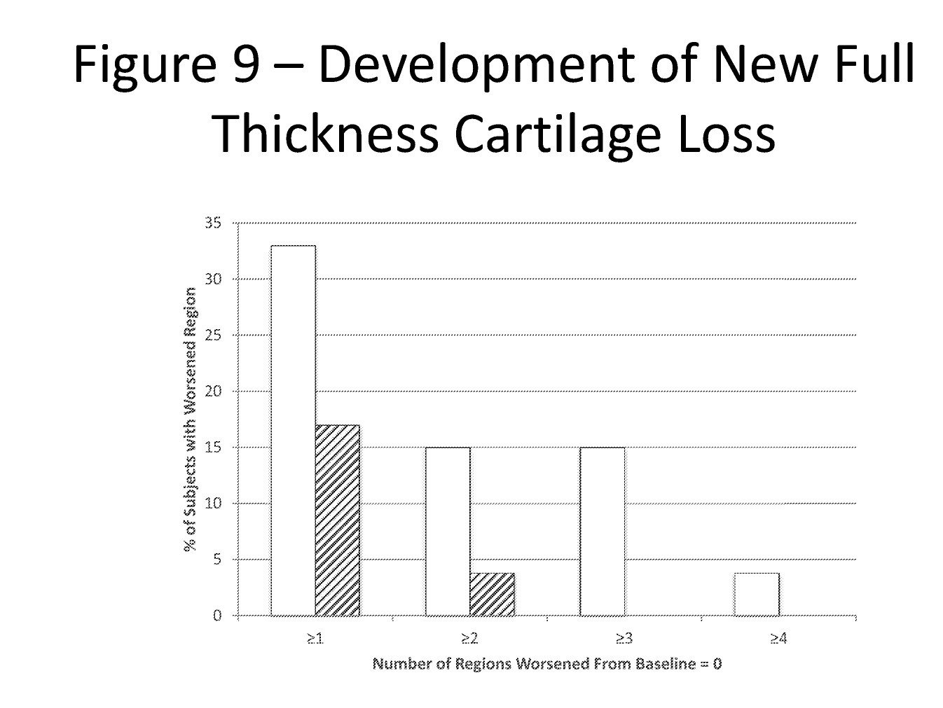

FIG. 9 shows that treatment was associated with a reduction in the incidence of patients who developed full thickness cartilage loss in regions that had no full thickness cartilage loss loss at baseline.

These data demonstrate that treatment with adipose-derived regenerative cells reduces the incidence of worsening of cartilage loss in damaged joints, e.g., worsening of progression of cartilage loss or the development of cartilage loss where there was no previous cartilage loss observed. Furthermore, adipose-derived regenerative cells advantageously significantly reduced the worsening of cartilage loss in the central/medial regions of the knee.

### Example 2—Preventing and/or Slowing Formation of Osteophytes

A total of 94 patients with osteoarthritis of the knee were enrolled in a double-blind, placebo-controlled, dose escalation study to study the effects of regenerative cell therapy on various aspects of osteoarthritis. Patients with diagnosis of osteoarthritis in one or both knees by the American College of Rheumatology criteria, with imaging findings of degenerative changes in the joint were eligible for the study. All patients underwent small volume liposuction under local anesthesia. The collected lipoaspirate was processed in the Cytori CELUTION® cell processing system to isolate and concentrate adipose-derived regenerative cells for immediate intraartiular administration.

After cell processing, patients were randomized 1:1:1 ratio into the following groups: placebo; low dose (20 million cells); and high dose (40 million cells).

At baseline and again at 48 weeks post-treatment, patients underwent an MRI. Osteophyte size was scored in the following regions of the knee was analyzed: femur lateral central region, femur lateral posterior region, femur medial central region, femur medial posterior region, femur trochlea lateral region, femur trochlea medial region, tibial subspinous region, patella interior region, patellar superior region, patella medial region, patella lateral region, tibia lateral region, and tibia medial region.

A radiologist blinded to treatment examined each region of the knee joint for osteophyte size. The radiologist scored the largest osteophyte within the specified region on a scale of 0 to 3 as follows:

0=none;

1=small;

2=medium;

3=large.

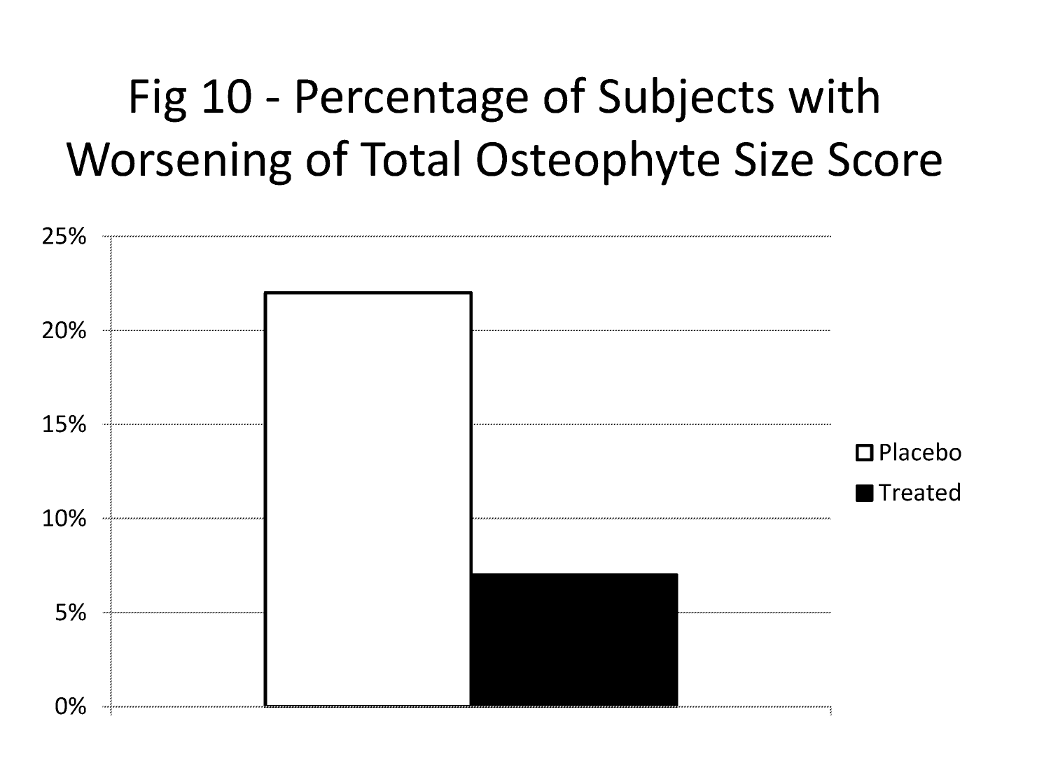

As shown in FIG. 10, a lower percentage of patients treated with adipose-derived regenerative cells exhibited worsening of total osteophyte score than patients treated with placebo.

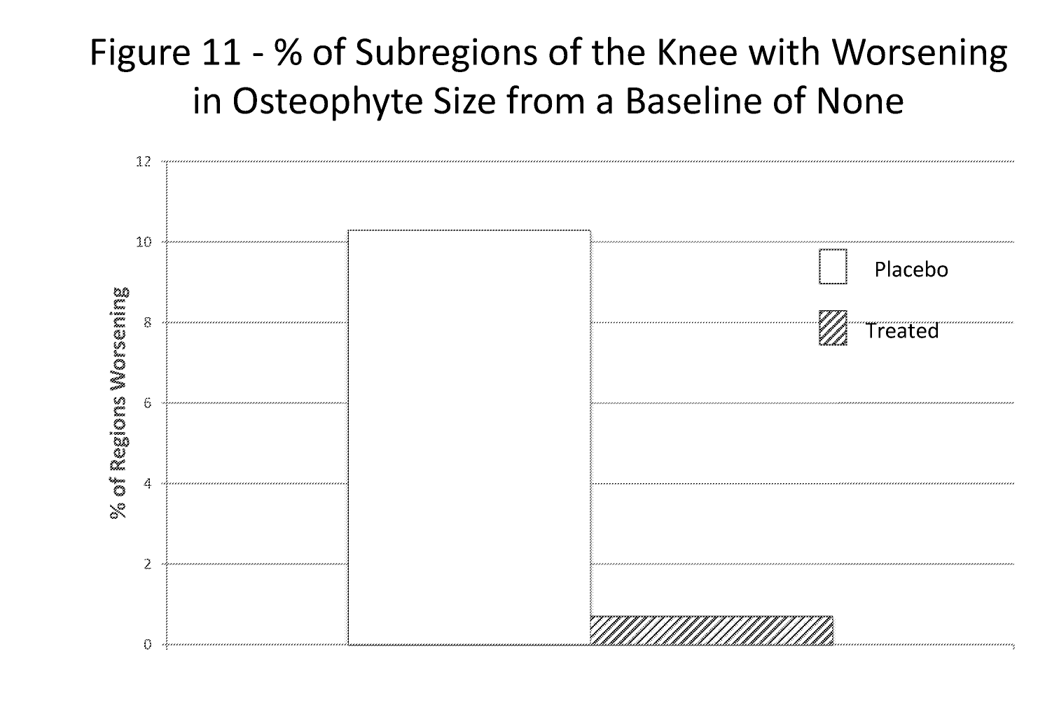

As shown in FIG. 11, a lower percentage of regions within patients treated with adipose-derived regenerative cells exhibited formation of osteophytes in regions that had no osteophytes at baseline than patients treated with placebo.

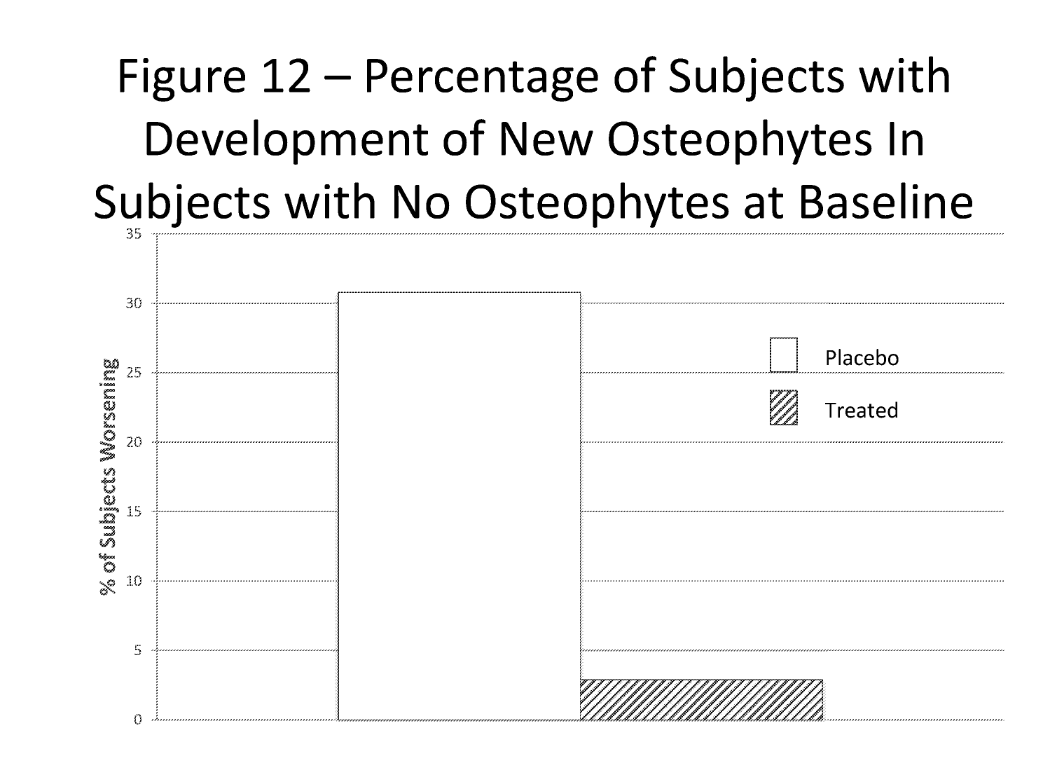

As shown in FIG. 12, a lower percentage of patients treated with adipose-derived regenerative cells exhibited formation of osteophytes in regions that had no osteophytes at baseline than patients treated with placebo.

The data demonstrate that adipose-derived regenerative cells are useful in preventing or slowing the formation of osteophytes in one or more regions of a joint, and further that treatment was associated with a reduction in the development of osteophytes in regions that had no osteophytes at baseline.

### Example 3—Preventing or Slowing the Formation of Bone Marrow Lesions

A total of 94 patients with osteoarthritis of the knee were enrolled in a double-blind, placebo-controlled, dose escalation study to study the effects of regenerative cell therapy on various aspects of osteoarthritis. Patients with diagnosis of osteoarthritis in one or both knees by the American College of Rheumatology criteria, with imaging findings of degenerative changes in the joint were eligible for the study. All patients underwent small volume liposuction under local anesthesia. The collected lipoaspirate was processed in the Cytori CELUTION® cell processing system to isolate and concentrate adipose-derived regenerative cells for immediate intraartiular administration.

After cell processing, patients were randomized 1:1:1 ratio into the following groups: placebo; low dose (20 million cells); and high dose (40 million cells).

At baseline and again at 48 weeks post-treatment, patients underwent an MRI. Bone marrow lesions analyzed in the following subregions of the knee were analyzed: femur lateral central region, femur lateral posterior region, femur medial central region, femur medial posterior region, femur trochlea lateral region, femur trochlea medial region, tibial subspinous region, patella interior region, patellar superior region, patella medial region, patella lateral region, tibia lateral region, and tibia medial region. The number of bone marrow lesions in each subregion was determined, and given a score of 0 to 10, with 0 corresponding to no bone marrow lesions in the subregion and 10 corresponding to 10 bone marrow lesions in the subregion. The size of bone marrow lesions (including volume of any associated cyst) as a percentage of the subregion volume was determined and scored as follows:

0=none

1=less than 33% of the subregion volume

2=33-66% of the subregion volume

3=greater than 66% of the subregion volume.

Table 2 shows that the percentage of persons with worsening of total scores (sum of changes over all regions) for different parameters for assessing bone marrow lesions was lower in subjects treated with adipose-derived regenerative cells compared to subjects treated with placebo.

| | Total BML | Total BML size as a | Total Number |
|---|---|---|---|
| Arm | not Cyst | % of surface area | of BMLs |
| Placebo | 44% (12/27) | 56% (15/27) | 52% (14/27) |
| ADRC treatment | 38% (20/53) | 36% (19/53) | 42% (22/52) |

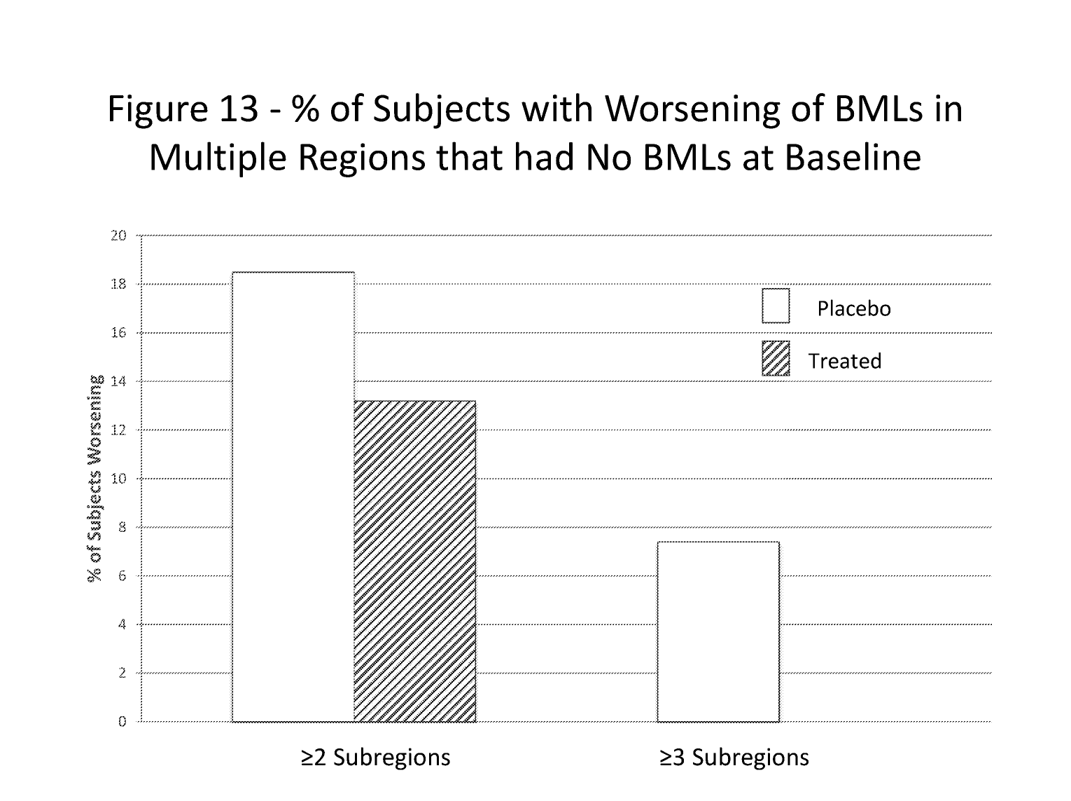

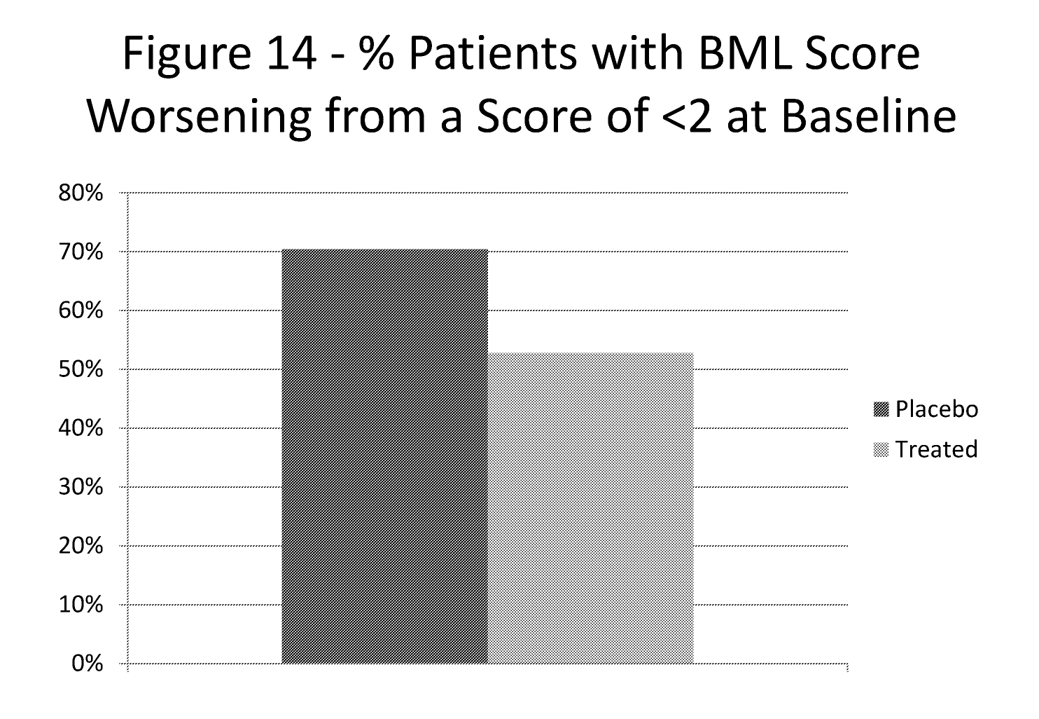

As shown in FIG. 13, treatment with adipose-derived regenerative cells reduced the development of bone marrow lesions in multiple regions that had no bone marrow lesions at baseline. FIG. 14 shows that treatment with adipose-derived regenerative cells reduced the incidence of any region with a bone marrow lesion size or percentage not cyst progressing from a score of less than 2, to a score of 2 or greater than 2. It has been reported that patients with a bone marrow lesion score of ≥2 exhibit an increased rate of progression of osteoarthritis (increased rate of joint space narrowing) See, Edwards et al., (2016) J Rheumatol 43(3) 657-65)

The data demonstrate that adipose-derived regenerative cells slow the worsening of bone marrow lesions (e.g., size, % BML, that is non-cyst, number of BMLs); and prevent the formation of bone marrow lesions.

### EQUIVALENTS

The compositions and methods disclosed herein are not to be limited in scope by the specific embodiments described herein. Indeed, various modifications of the compositions and methods in addition to those described will become apparent to those skilled in the art from the foregoing description. Such modifications are intended to fall within the scope of the appended claims.

Various publications, patents and patent applications are cited herein, the disclosures of which are incorporated by reference in their entireties.

1. A method of reducing the worsening of cartilage loss in at least one region of a joint in a subject in need thereof, comprising:
identifying a subject at risk of developing new or worsened cartilage loss in at least one region of a joint; and
administering to the identified subject a therapeutically effective amount of regenerative cells.

2-59. (canceled)

60. The method of claim 1, wherein the joint is selected from the group consisting of a knee, a hand, a hip, an elbow, a finger, a spine, an ankle, and a toe.

61. The method of claim 60, wherein the joint is a knee.

62. The method of claim 61, wherein the region of the knee joint is selected from the group consisting of: femur lateral central region, femur lateral posterior region, femur medial posterior region, femur trochlea lateral region, femur trochlea medial region, patella lateral region, patella medial region, tibial lateral anterior region, tibial lateral central region, tibia lateral posterior region, tibia medial anterior region, tibia medial central region, and tibia medial posterior region.

63. The method of claim 1, wherein the reduction of the worsening of cartilage loss in at least one region of a joint comprises reduction of the worsening of full thickness cartilage loss.

64. The method of claim 1, wherein the reduction of the worsening of cartilage loss in at least one region of a joint comprises reduction of the worsening of partial thickness cartilage loss.

65. The method of claim 1, wherein the identified subject has at least one region of the joint with no full thickness cartilage loss prior to administration of the regenerative cells.

66. The method of claim 1, wherein the identified subject has a region at risk of development of full thickness cartilage loss, which has less than 10% full thickness cartilage loss prior to administration of the regenerative cells.

67. The method of claim 1, wherein the identified subject has a region at risk of development of full thickness cartilage loss, which has between 10 to 75% full thickness cartilage loss prior to administration of the regenerative cells.

68. The method of claim 1, wherein the identified subject has a region at risk of development of full thickness cartilage loss, which has greater than 75% full thickness cartilage loss prior to administration of the regenerative cells.

69. The method of claim 1, wherein the subject has at least one region of the joint with no partial thickness cartilage loss prior to administration of the regenerative cells.

70. The method of claim 1, wherein the identified subject has a region at risk of development of partial thickness cartilage loss, which has less than 10% partial thickness cartilage loss prior to administration of the regenerative cells.

71. The method of claim 1, wherein the identified subject has a region at risk of development of partial thickness cartilage loss, which has between 10 to 75% partial thickness cartilage loss prior to administration of the regenerative cells.

72. The method of claim 1, wherein the identified subject has a region at risk of development of partial thickness cartilage loss, which has greater than 75% partial thickness cartilage loss prior to administration of the regenerative cells.

73. A method of preventing or slowing the formation and/or growth of osteophytes in one or more regions of a joint of a subject, comprising:
identifying a subject having, or at risk of developing, one or more osteophytes in one or more regions of a joint; and
administering to the identified subject a therapeutically effective amount of regenerative cells.

74. The method of claim 73, further comprising assessing osteophytes in the subject.

75. The method of claim 73, wherein the one or more regions of joint has no osteophytes prior to administration of the therapeutically effective amount of regenerative cells.

76. The method of claim 73, wherein the joint is selected from the group consisting of: a knee, a hand, a hip, an elbow, a finger, a spine, an ankle, and a toe.

77. The method of claim 1, wherein the regenerative cells are adipose-derived regenerative cells that have not been cultured prior to administration to the subject.

78. The method of claim 73, wherein the regenerative cells are adipose-derived regenerative cells that have not been cultured prior to administration to the subject.
